(19) United States
(12) Patent Application Publication
(10) Pub. No.: US 2022/0219154 A1
(43) Pub. Date: Jul. 14, 2022

(54) MOLDING COMPRISING A TYPE MFI ZEOLITIC TITANOSILICATE AND A SILICA BINDER, ITS PREPARATION PROCESS AND USE AS CATALYST

(71) Applicant: BASF SE, Ludwigshafen am Rhein (DE)

(72) Inventors: Andrei-Nicolae PARVULESCU, Ludwigshafen am Rhein (DE); Hans-Juergen LUETZEL, Boehl-lggelheim (DE); Dominic RIEDEL, Ludwigshafen am Rhein (DE); Ulrich MUELLER, Ludwigshafen am Rhein (DE); Joaquim Henrique TELES, Ludwigshafen am Rhein (DE); Markus WEBER, Ludwigshafen am Rhein (DE)

(21) Appl. No.: 17/606,875

(22) PCT Filed: Apr. 27, 2020
(86) PCT No.: PCT/EP2020/061597

§ 371 Date: Oct. 27, 2021

(30) Foreign Application Priority Data
Apr. 29, 2019 (EP) 19171503.6

(51) Int. Cl. B01J 29/89; B01J 35/10; B01J 35/00; B01J 35/02; B01J 35/04; B01J 37/00; B01J 37/08

(52) U.S. Cl. CPC B01J 29/89; B01J 35/1042; B01J 35/002; B01J 35/023; B01J 2229/42; B01J 37/0009; B01J 37/08; B01J 2229/36; B01J 35/04

(57) ABSTRACT

A chemical molding comprising a zeolitic material which exhibits a type I nitrogen adsorption/desorption isotherm determined as described in Reference Example 1, and which has framework type MFI and a framework structure comprising Si, O, and Ti, the molding further comprising a binder for said zeolitic material, the binder comprising Si and O, wherein the molding exhibits a total pore volume of at least 0.4 mL/g and a crushing strength of at least 6 N.

The present invention relates to a chemical molding particularly comprising a specific binder and a specific zeolitic material which has framework type MFI and a framework structure comprising Si, O, and Ti.

Titanium containing zeolitic materials of structure type MFI, exhibiting a type I nitrogen adsorption/desorption isotherm, such as titanium silicalite-1, are known to be efficient catalysts including, for example, epoxidation reactions. In such industrial-scale processes, typically carried out in continuous mode, these zeolitic materials are usually employed in the form of moldings which, in addition to the catalytically active zeolitic material, comprise a suitable binder.

US 2016/250624 A1 relates to a process for the production of a molding containing hydrophobic zeolitic materials, and to a process for the preparation thereof.

U.S. Pat. No. 6,551,546 B1 relates to a process for producing a shaped body comprising at least one porous oxidic material and at least one metal oxide.

DE 19859561 A1 similarly relates to a process for preparing a shaped body comprising at least one porous oxidic material and at least one metal oxide.

U.S. Pat. No. 7,825,204 B2 relates to an extrudate comprising an inorganic oxide and a comb-branched polymer is disclosed.

It was an object of the present invention to provide a novel and advantageous molding comprising a zeolitic material having framework type MFI having advantageous characteristics, in particular an improved propylene oxide selectivity when used as a catalyst or catalyst component, in particular in the epoxidation reaction of propene to propylene oxide. It was a further object of the present invention to provide a process for the preparation of such a molding, in particular to provide a process resulting in a molding having advantageous properties, preferably when used as a catalyst or catalyst component, specifically in an oxidation or epoxidation reaction. It was a further object of the present invention to provide an improved process for the epoxidation of propene with hydrogen peroxide as oxidizing agent, exhibiting a very low selectivity with respect to by-products and side-products of the epoxidation reaction while, at the same time, allowing for a very high propylene selectivity.

Surprisingly, it was found that such a molding exhibiting said advantageous characteristics can be provided if, for preparing the moldings, a specific binder precursor material given is used, and an intermediate molding comprising a zeolitic material having framework type MFI is subjected to a specific post-treatment. In particular, it has surprisingly been found that a molding can be provided which shows, if used as a catalyst in an epoxidation reaction of propene to propylene oxide and if compared to prior art moldings, significantly increased propylene oxide selectivity and yield, and further exhibits excellent life time properties.

Therefore, the present invention relates to a chemical molding comprising a zeolitic material which exhibits a type I nitrogen adsorption/desorption isotherm and which has framework type MFI and a framework structure comprising Si, O, and Ti, the molding further comprising a binder for said zeolitic material, the binder comprising Si and O, wherein the molding exhibits a total pore volume of at least 0.4 mL/g and a crushing strength of at least 6 N. In particular, the present invention relates to a chemical molding comprising a zeolitic material which exhibits a type I nitrogen adsorption/desorption isotherm determined as described in Reference Example 1, and which has framework type MFI and a framework structure comprising Si, O, and Ti, the molding further comprising a binder for said zeolitic material, the binder comprising Si and O, wherein the molding exhibits a total pore volume of at least 0.4 mL/g, determined as described in Reference Example 2, and a crushing strength of at least 6 N, determined as described in Reference Example 3.

According to the present invention, a molding is to be understood as a three-dimensional entity obtained from a shaping process; accordingly, the term “molding” is used synonymously with the term “shaped body”.

Further, the present invention relates to a process for preparing a chemical molding comprising a zeolitic material which exhibits a type I nitrogen adsorption/desorption isotherm, determined as described in Reference Example 1, and which has framework type MFI and a framework structure comprising Si, O, and Ti, the molding further comprising a binder for said zeolitic material, the binder comprising Si and O, preferably for preparing an inventive chemical molding as described herein, the process comprising
(i) providing a zeolitic material exhibiting a type I nitrogen adsorption/desorption isotherm, determined as described in Reference Example 1, having framework type MFI and a framework structure comprising Si, O, and Ti;
(ii) providing a binder precursor comprising a colloidal dispersion of silica in water, said binder precursor exhibiting a volume-based particle size distribution characterized by a Dv10 value of at least 35 nanometer, a Dv50 value of at least 45 nanometer, and a Dv90 value of at least 65 nanometer, determined as described in Reference Example 5;
(iii) preparing a mixture comprising the zeolitic material provided in (i) and the binder precursor provided in (ii);
(iv) shaping the mixture obtained from (iii), obtaining a precursor of the molding;
(v) preparing a mixture comprising the precursor of the molding obtained from (iv) and water, and subjecting the mixture to a water treatment under hydrothermal conditions, obtaining a water-treated precursor of the molding;
(vi) calcining the water-treated precursor of the molding in a gas atmosphere, obtaining the molding.

Yet further, the present invention relates to a chemical molding comprising particles of a zeolitic material exhibiting a type I nitrogen adsorption/desorption isotherm, determined as described in Reference Example 1, having framework type MFI and a framework structure comprising Si, O, and Ti, the molding further comprising a binder for said particles, the binder comprising Si and O, preferably a chemical molding obtainable or obtained by the inventive process as described herein.

Yet further, the present invention relates to a use of an inventive molding as described herein as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component.

Yet further, the present invention relates to a process for oxidizing an organic compound comprising bringing the organic compound in contact, preferably in continuous mode, with a catalyst comprising a molding as described herein, preferably for epoxidizing an organic compound, more preferably for epoxidizing an organic compound having at least one C—C double bond, preferably a C2-C10 alkene, more preferably a C2-C5 alkene, more preferably a C2-C4 alkene, more preferably a C2 or C3 alkene, more preferably propene.

Yet further, the present invention relates to a process for preparing propylene oxide comprising reacting propene, preferably in continuous mode, with hydrogen peroxide in methanolic solution in the presence of a catalyst comprising a molding as described herein to obtain propylene oxide.

Yet further, the present invention relates to a use of a colloidal dispersion of silica in water as a binder precursor for preparing a chemical molding comprising a zeolitic material which exhibits a type I nitrogen adsorption/desorption isotherm, determined as described in Reference Example 1, and which has framework type MFI and a framework structure comprising Si, O, and Ti, the molding further comprising a binder resulting from said binder precursor, preferably for preparing the molding as described herein, said silica exhibiting a volume-based particle size distribution characterized by a Dv10 value of at least 35 nanometer, preferably in the range of from 35 to 80 nanometer, more preferably in the range of from 40 to 75 nanometer, more preferably in the range of from 45 to 70 nanometer, a Dv50 value of at least 45 nanometer, preferably in the range of from 45 to 125 nanometer, more preferably in the range of from 55 to 115 nanometer, more preferably in the range of from 65 to 105 nanometer, and a Dv90 value of at least 65 nanometer, preferably in the range of from 65 to 200 nanometer, more preferably in the range of from 85 to 180 nanometer, more preferably in the range of from 95 to 160 nanometer, determined as described in Reference Example 5, said molding preferably exhibiting a total pore volume of at least 0.4 mL/g, determined as described in Reference Example 2, and a crushing strength of at least 6 N, determined as described in Reference Example 3.

As regards the inventive chemical molding, it is preferred that from 95 to 100 weight-%, preferably from 98 to 100 weight-%, more preferably from 99 to 100 weight-%, more preferably from least 99.5 to 100 weight-%, more preferably from 99.9 to 100 weight-% of the zeolitic material comprised in the molding consist of Si, O, Ti and optionally H.

As regards the zeolitic material comprised in the chemical molding, it is preferred that the zeolitis material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the zeolitic material.

Further, it is preferred that the zeolitic material comprised in the molding is titanium silicalite-1.

As regards the binder, it is preferred that from 95 to 100 weight-%, preferably from 98 to 100 weight-%, more preferably from 99 to 100 weight-%, more preferably from at least 99.5 to 100 weight-%, more preferably from 99.9 to 100 weight-% of the binder comprised in the molding consist of Si and O.

It is preferred that the molding comprises the binder, calculated as SiO₂, in an amount in the range of from 2 to 90 weight-%, more preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the molding.

Further, it is preferred that from 95 to 100 weight-%, more preferably from 98 to 100 weight-%, more preferably from 99 to 100 weight-%, more preferably from least 99.5 to 100 weight-%, more preferably from 99.9 to 100 weight-% of the molding consist of the zeolitic material and the binder.

It is preferred that the molding comprises micropores having a pore diameter in the range of from 0.1 to less than 2 nm, determined as described in Reference Example 4. Further, it is preferred that the molding comprises mesopores having a pore diameter in the range of from 2 to 50 nm, determined as described in Reference Example 4. Thus, it is particularly preferred that the molding comprises micropores having a pore diameter in the range of from 0.1 to less than 2 nm, determined as described in Reference Example 4 and mesopores having a pore diameter in the range of from 2 to 50 nm, determined as described in Reference Example 4.

Preferably, the molding as disclosed herein exhibits a total pore volume in the range of from 0.4 to 1.5 mL/g, more preferably in the range of from 0.4 to 1.2 mL/g, more preferably in the range of from 0.4 to 1.0 mL/g, wherein the pore volume is determined as described in Reference Example 2.

Further, it is preferred that the molding as disclosed herein exhibits a crushing strength in the range of from 6 to 25 N, more preferably in the range of from 7 to 20 N, more preferably in the range of from 8 to 15 N, wherein the crushing strength is determined as described in Reference Example 3.

It is preferred that the molding is a strand. It is particularly preferred that the molding being a strand has a hexagonal, rectangular, quadratic, triangular, oval, or circular cross-section, more preferably a circular cross-section. It is particularly preferred that the molding being a strand is an extrudate.

In the case where the molding is a strand having a circular cross-section, it is preferred that the cross-section has a diameter in the range of from 0.5 to 5 mm, more preferably in the range of from 1 to 3 mm, more preferably in the range of from 1.5 to 2 mm. It is particularly preferred that the molding being a strand having a circular cross-section with a specific diameter as disclosed herein is an extrudate.

Thus, it is preferred that the molding as disclosed herein is an extrudate.

It is preferred that the molding exhibits a tortuosity parameter relative to water in the range of from 1.0 to 2.5, more preferably in the range of from 1.3 to 2.0, more preferably in the range of from 1.6 to 1.8, more preferably in the range of from 1.6 to 1.75, more preferably in the range of from 1.6 to 1.72, determined as described in Reference Example 11.

Further, it is preferred that the molding exhibits a BET specific surface area in the range of from 300 to 450 m²/g, more preferably in the range of from 310 to 400 m²/g, more preferably in the range of from 320 to 375 m²/g, determined as described in Reference Example 6.

As regards the crystallinity of the molding, it is preferred that the molding exhibits a crystallinity in the range of from 50 to 100%, more preferably in the range of from 50 to 90%, more preferably in the range of from 50 to 80%, determined as described in Reference Example 7.

As regards the propylene oxide activity of the molding it is preferred that the molding of exhibits a propylene oxide activity of at least 4.5 weight-%, more preferably in the range of from 4.5 to 11 weight-%, more preferably in the range of from 4.5 to 10 weight-%, determined as described in Reference Example 9.

It is preferred that the molding exhibits a pressure drop rate in the range of from 0.005 to 0.019 bar(abs)/min, more preferably in the range of from 0.006 to 0.017 bar(abs)/min, more preferably in the range of from 0.007 to 0.015 bar(abs)/min, determined as described in Reference Example 9.

Preferably, the molding is used as catalyst or catalyst component, in particular in a reaction for preparing propylene oxide from propene and hydrogen peroxide. In this regard, it is preferred that the molding being used as catalyst in a reaction for preparing propylene oxide from propene and hydrogen peroxide, preferably in a continuous epoxidation reaction as described in Reference Example 10, exhibits a hydrogen peroxide conversion in the range of from 90 to 95%, wherein preferably the temperature of the cooling medium is in the range of from 55 to 56° C. and the time on stream is in the range of from 200 to 600 hours, preferably time on stream is in the range of from 300 to 600 hours, more preferably the time on stream is in the range of from 350 to 600 hours. In this regard, the term “time on stream” refers to the duration of the continuous epoxidation reaction without regeneration of the catalyst.

Further, the present invention relates to a process for preparing a chemical molding comprising a zeolitic material which exhibits a type I nitrogen adsorption/desorption isotherm, determined as described in Reference Example 1, and which has framework type MFI and a framework structure comprising Si, O, and Ti, the molding further comprising a binder for said zeolitic material, the binder comprising Si and O, preferably for preparing the chemical molding as described herein, the process comprising
(i) providing a zeolitic material exhibiting a type I nitrogen adsorption/desorption isotherm, determined as described in Reference Example 1, having framework type MFI and a framework structure comprising Si, O, and Ti;
(ii) providing a binder precursor comprising a colloidal dispersion of silica in water, said binder precursor exhibiting a volume-based particle size distribution characterized by a Dv10 value of at least 35 nanometer, a Dv50 value of at least 45 nanometer, and a Dv90 value of at least 65 nanometer, determined as described in Reference Example 5;
(iii) preparing a mixture comprising the zeolitic material provided in (i) and the binder precursor provided in (ii);
(iv) shaping the mixture obtained from (iii), obtaining a precursor of the molding;
(v) preparing a mixture comprising the precursor of the molding obtained from (iv) and water, and subjecting the mixture to a water treatment under hydrothermal conditions, obtaining a water-treated precursor of the molding;
(vi) calcining the water-treated precursor of the molding in a gas atmosphere, obtaining the molding.

It is preferred that the volume-based particle size distribution of the colloidal dispersion of silica in water according to (ii) is characterized by a Dv10 value in the range of from 35 to 80 nanometer, more preferably in the range of from 40 to 75 nanometer, more preferably in the range of from 45 to 70 nanometer, a Dv50 value in the range of from 45 to 125 nanometer, more preferably in the range of from 55 to 115 nanometer, more preferably in the range of from 65 to 105 nanometer, and a Dv90 value in the range of from 65 to 200 nanometer, more preferably in the range of from 85 to 180 nanometer, more preferably in the range of from 95 to 160 nanometer, determined as described in Reference Example 5.

Further, it is preferred that the volume-based particle size distribution of the colloidal dispersion of silica in water according to (ii) is a mono-modal distribution.

As regards the content of silica comprised in the colloidal dispersion of silica in water according to (ii), no particular restriction applies. It is preferred that the colloidal dispersion of silica in water according to (ii) comprises the silica in an amount in the range of from 25 to 65 weight-%, more preferably in the range of from 30 to 60 weight-%, more preferably in the range of from 35 to 55 weight-%, based on the total weight of the silica and the water.

It is preferred that from 95 to 100 weight-%, more preferably from 98 to 100 weight-%, more preferably from 99 to 100 weight-% of the binder precursor according to (ii) consist of the colloidal dispersion of silica in water.

Further, it is preferred that from 95 to 100 weight-%, more preferably from 98 to 100 weight-%, more preferably from 99 to 100 weight-%, more preferably from least 99.5 to 100 weight-%, more preferably from 99.9 to 100 weight-% of the zeolitic material according to (i) consist of Si, O, Ti and preferably H.

As regards the content of Ti in the zeolitic material according to (i), no particular restriction applies. It is preferred that the zeolitic material according to (i) comprises Ti in an amount in the range of from 0.2 to 5 weight-%, more preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, based on the total weight of the zeolitic material.

It is preferred that the zeolitic material according to (i) is titanium silicalite-1.

Further, it is preferred that in the mixture prepared according to (iii) and subjected to (iv), the weight ratio of the zeolitic material, relative to the sum of the zeolitic material and the binder calculated as SiO₂, is in the range of from 2 to 90%, more preferably in the range of from 5 to 70%, more preferably in the range of from 10 to 50%, more preferably in the range of from 15 to 30%, more preferably in the range of from 20 to 25%.

The mixture disclosed herein may comprise further components. It is preferred that the mixture prepared according to (iii) and subjected to (iv) further comprises one or more additives, more preferably one or more viscosity modifying agents, or one or more mesopore forming agents, or one or more viscosity modifying agents and one or more mesopore forming agents.

In the case where the mixture prepared according to (iii) and subjected to (iv) further comprises one or more additives, it is preferred that from 95 to 100 weight-%, more preferably from 98 to 100 weight-%, more preferably from 99 to 100 weight-%, more preferably from 99.5 to 100 weight-%, more preferably from 99.9 to 100 weight-% of the mixture prepared according to (iii) and subjected to (iv) consist of the zeolitic material, the binder precursor, and the one or more additives.

Further in the case where the mixture prepared according to (iii) and subjected to (iv) further comprises one or more additives, it is preferred that the one or more additives are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose ethers, polyalkylene oxides, polystyrenes, and mixtures of two or more thereof, wherein the organic polymers are more preferably selected from the group consisting of a methyl celluloses, carboxymethyl celluloses, polyethylene oxides, polystyrenes, and mixtures of two or more thereof, wherein more preferably, the one or more additives comprise, more preferably consist of, water, a carboxymethyl cellulose, a polyethylene oxide, and a polystyrene.

Further in the case where the mixture prepared according to (iii) and subjected to (iv) further comprises one or more additives, it is preferred that in the mixture prepared according to (iii) and subjected to (iv), the weight ratio of the zeolitic material, relative to the one or more additives, is in the range of from 0.3:1 to 1:1, more preferably in the range of from 0.4:1 to 0.8:1, more preferably in the range of from 0.5:1 to 0.6:1.

In the case where the mixture prepared according to (iii) and subjected to (iv) further comprises a cellulose derivative as additive, it is preferred that in the mixture prepared according to (iii) and subjected to (iv), the weight ratio of the zeolitic material, relative to the cellulose derivative, preferably a cellulose ether, more preferably carboxymethyl cellulose, is in the range of from 10:1 to 53:1, more preferably in the range of from 15:1 to 40:1, more preferably in the range of from 20:1 to 35:1.

In the case where the mixture prepared according to (iii) and subjected to (iv) further comprises a polyethylene oxide as additive, it is preferred that in the mixture prepared according to (iii) and subjected to (iv), the weight ratio of the zeolitic material, relative to the polyethylene oxide, is in the range of from 70:1 to 110:1, more preferably in the range of from 75:1 to 100:1, more preferably in the range of from 77:1 to 98:1.

In the case where the mixture prepared according to (iii) and subjected to (iv) further comprises a polystyrene as additive, it is preferred that in the mixture prepared according to (iii) and subjected to (iv), the weight ratio of the zeolitic material, relative to the polystyrene, is in the range of from 2:1 to 8:1, more preferably in the range of from 3:1 to 6:1, more preferably in the range of from 3.5:1 to 5:1.

In the case where the mixture prepared according to (iii) and subjected to (iv) further comprises water as additive, it is preferred that in the mixture prepared according to (iii) and subjected to (iv), the weight ratio of the zeolitic material, relative to the water, is in the range of from 0.7:1 to 0.85:1, more preferably in the range of from 0.72:1 to 0.8:1, more preferably in the range of from 0.74:1 to 0.0.79:1.

It is particularly preferred that the mixture prepared according to (iii) and subjected to (iv) further comprises a cellulose derivative, a polyethylene oxide, a polystyrene, and water as additives.

As regards the provision of the mixture in (iii), i.e. the method how the mixture is prepared, no particular restrictions applies. It is preferred that the mixture is prepared by suitably mixing the respective components, preferably by mixing in a kneader or in a mix-muller.

Further, it is preferred that according to (iv), the mixture obtained from (iii) is shaped to a strand, more preferably to a strand having a circular cross-section, wherein the strand having a circular cross-section has a diameter preferably in the range of from 0.5 to 5 mm, more preferably in the range of from 1 to 3 mm, more preferably in the range of from 1.5 to 2 mm.

Further, it is preferred that the mixture obtained from (iii) and subjected to (iv) has a plasticity in the range of from 500 to 3000 N, more preferably in the range of from 750 to 2000 N, more preferably in the range of from 1000 to 1500 N, determined as described in Reference Example 12.

As regards shaping in (iv), no particular restriction applies such that shaping may be performed by any conceivable means. It is preferred that shaping according to (iv) comprises extruding the mixture obtained from (iii).

Suitable extrusion apparatuses are described, for example, in “Ullmann's Enzyklopädie der Technischen Chemie”, 4th edition, vol. 2, page 295 et seq., 1972. In addition to the use of an extruder, an extrusion press can also be used for the preparation of the moldings. If necessary, the extruder can be suitably cooled during the extrusion process. The strands leaving the extruder via the extruder die head can be mechanically cut by a suitable wire or via a discontinuous gas stream.

The shaping according to (iv) may comprise further process steps. It is preferred that shaping according to (iv) further comprises drying the precursor of the molding in a gas atmosphere, wherein said drying is preferably carried out at a temperature of the gas atmosphere in the range of from 80 to 160° C., more preferably in the range of from 100 to 140° C., more preferably in the range of from 110 to 130° C., wherein the gas atmosphere preferably comprises nitrogen, oxygen, or a mixture thereof, wherein the gas atmosphere is more preferably oxygen, air, or lean air.

Further, it is preferred that shaping according to (iv) further comprises calcining the preferably dried precursor of the molding in a gas atmosphere, wherein calcining is preferably carried out at a temperature of the gas atmosphere in the range of from 450 to 530° C., more preferably in the range of from 470 to 510° C., more preferably in the range of from 480 to 500° C., wherein the gas atmosphere comprises preferably nitrogen, oxygen, or a mixture thereof, wherein the gas atmosphere is more preferably oxygen, air, or lean air.

As regards the content of water in the mixture prepared in (v), no particular restriction applies. It is preferred that in the mixture prepared in (v), the weight ratio of the precursor of the molding relative to the water is in the range of from 1:1 to 1:30, more preferably in the range of from 1:5 to 1:25, more preferably in the range of from 1:10 to 1:20.

Further, it is preferred that from 95 to 100 weight-%, more preferably from 98 to 100 weight-%, more preferably from 99 to 100 weight-%, more preferably from 99.5 to 100 weight-%, more preferably from 99.9 to 100 weight-% of the mixture prepared according to (v) consist of the precursor of the molding and water.

As regards the temperature of the mixture for the the water treatment according to (v), no particular restriction applies. It is preferred that the water treatment according to (v) comprises a temperature of the mixture in the range of from 100 to 200° C., more preferably in the range of from 125 to 175° C., more preferably in the range of from 130 to 160° C., more preferably in the range of from 135 to 155° C. more preferably in the range of from 140 to 150° C.

It is preferred that the water treatment according to (v) is carried out under autogenous pressure, preferably in an autoclave.

It is preferred that the water treatment according to (v) is carried out for 6 to 10 h, more preferably for 7 to 9 h, more preferably for 7.5 to 8.5 h.

Further, it is preferred that (v) further comprises separating the water-treated precursor of the molding from the mixture obtained from the water treatment.

In the case where (v) further comprises separating the water-treated precursor of the molding from the mixture obtained from the water treatment, it is preferred that separating the water-treated precursor of the molding from the mixture obtained from the water treatment comprises subjecting the mixture obtained from the water treatment to solid-liquid separation, preferably washing the separated precursor, and preferably drying the preferably washed precursor.

Further, in the case where separating the water-treated precursor of the molding from the mixture obtained from the water treatment comprises subjecting the mixture obtained from the water treatment to solid-liquid separation, it is preferred that the solid-liquid separation according to (v) comprises filtration, or centrifugation, or filtration and centrifugation.

In the case where (v) comprises washing the separated precursor, it is preferred that washing the precursor is conducted at least once with a liquid solvent system, wherein the liquid solvent system preferably comprises one or more of water, an alcohol, and a mixture of two or more thereof, wherein the water-treated precursor of the molding is more preferably washed with water.

In the case where (v) further comprises drying the preferably washed precursor, it is preferred that drying according to (v) comprises drying the precursor in a gas atmosphere, wherein drying is more preferably carried out at a temperature of the gas atmosphere in the range of from 80 to 160° C., more preferably in the range of from 100 to 140° C., more preferably in the range of from 110 to 130° C., wherein the gas atmosphere preferably comprises nitrogen, oxygen, or a mixture thereof, wherein the gas atmosphere is more preferably oxygen, air or lean air.

As regards the temperature of the gas atmosphere for the calcining according to (vi), no particular restriction applies. It is preferred that calcining according to (vi) is carried out at a temperature of the gas atmosphere in the range of from 400 to 490° C., more preferably in the range of from 420 to 470° C., more preferably in the range of from 440 to 460° C., wherein the gas atmosphere preferably comprises nitrogen, oxygen, or a mixture thereof, wherein the gas atmosphere is more preferably oxygen, air or lean air.

It is preferred that the inventive process as described herein consists of (i), (ii), (iii), (iv), (v) and (vi).

Further, the present invention relates to a chemical molding comprising particles of a zeolitic material exhibiting a type I nitrogen adsorption/desorption isotherm, determined as described in Reference Example 1, having framework type MFI and a framework structure comprising Si, O, and Ti, the molding further comprising a binder for said particles, the binder comprising Si and O, preferably the chemical molding as described herein, obtainable or obtained by the process as described herein.

Yet further, the present invention relates to a use of a molding as described herein as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component.

It is preferred that the inventive molding as described herein is used as an oxidation catalyst or as an oxidation catalyst component, more preferably as an epoxidation catalyst or as an epoxidation catalyst component, more preferably as an epoxidation catalyst.

In the case where the molding according to the present invention is used as an oxidation catalyst or as an oxidation catalyst component, the molding is preferably used for the epoxidation reaction of an organic compound having at least one C—C double bond, preferably a C2-C10 alkene, more preferably a C2-C5 alkene, more preferably a C2-C4 alkene, more preferably a C2 or C3 alkene, more preferably propene, more preferably for the epoxidation of propene with hydrogen peroxide as oxidizing agent, more preferably for the epoxidation of propene with hydrogen peroxide as oxidizing agent in a solvent comprising an alcohol, preferably methanol.

Yet further, the present invention relates to a process for oxidizing an organic compound comprising bringing the organic compound in contact, preferably in continuous mode, with a catalyst comprising a molding according to the present invention, preferably for epoxidizing an organic compound, more preferably for epoxidizing an organic compound having at least one C—C double bond, preferably a C2-C10 alkene, more preferably a C2-C5 alkene, more preferably a C2-C4 alkene, more preferably a C2 or C3 alkene, more preferably propene.

It is preferred that hydrogen peroxide is used as oxidizing agent, wherein the oxidation reaction is preferably carried out in a solvent, more preferably in a solvent comprising an alcohol, preferably methanol.

According to the present invention, it is conceivable that if hydrogen peroxide is used as oxidizing agent, the hydrogen peroxide is formed in situ during the reaction from hydrogen and oxygen or from other suitable precursors. More preferably, the term “using hydrogen peroxide as oxidizing agent” or similar as used in the context of the present invention relates to an embodiment where hydrogen peroxide is not formed in situ but employed as starting material, preferably in the form of a solution, preferably an at least partially aqueous solution, more preferably an aqueous solution, said preferably aqueous solution having a preferred hydrogen peroxide concentration in the range of from 20 to 60, more preferably from 25 to 55 weight-%, based on the total weight of the solution.

Yet further, the present invention relates to a process for preparing propylene oxide comprising reacting propene, preferably in continuous mode, with hydrogen peroxide in methanolic solution in the presence of a catalyst comprising a molding according to the present invention to obtain propylene oxide.

Yet further, the present invention relates to a use of a colloidal dispersion of silica in water as a binder precursor for preparing a chemical molding comprising a zeolitic material which exhibits a type I nitrogen adsorption/desorption isotherm, determined as described in Reference Example 1, and which has framework type MFI and a framework structure comprising Si, O, and Ti, the molding further comprising a binder resulting from said binder precursor, preferably for preparing a molding as described herein, said silica exhibiting a volume-based particle size distribution characterized by a Dv10 value of at least 35 nanometer, preferably in the range of from 35 to 80 nanometer, more preferably in the range of from 40 to 75 nanometer, more preferably in the range of from 45 to 70 nanometer, a Dv50 value of at least 45 nanometer, preferably in the range of from 45 to 125 nanometer, more preferably in the range of from 55 to 115 nanometer, more preferably in the range of from 65 to 105 nanometer, and a Dv90 value of at least 65 nanometer, preferably in the range of from 65 to 200 nanometer, more preferably in the range of from 85 to 180 nanometer, more preferably in the range of from 95 to 160 nanometer, determined as described in Reference Example 5, said molding preferably exhibiting a total pore volume of at least 0.4 mL/g, determined as described in Reference Example 2, and a crushing strength of at least 6 N, determined as described in Reference Example 3.

According to a further aspect, the present invention relates to a mixture comprising a zeolitic material which exhibits a type I nitrogen adsorption/desorption isotherm, determined as described in Reference Example 1, and which has framework type MFI and a framework structure comprising Si, O, and Ti, the mixture further comprising a colloidal dispersion of silica in water, said binder precursor exhibiting a volume-based particle size distribution characterized by a Dv10 value of at least 35 nanometer, a Dv50 value of at least 45 nanometer, and a Dv90 value of at least 65 nanometer, determined as described in Reference Example 5.

It is preferred that the mixture has a plasticity in the range of from 500 to 3000 N, more preferably in the range of from 750 to 2000 N, more preferably in the range of from 1000 to 1500 N, determined as described in Reference Example 12.

Further, it is preferred that the volume-based particle size distribution of the colloidal dispersion of silica in water is characterized by a Dv10 value in the range of from 35 to 80 nanometer, more preferably in the range of from 40 to 75 nanometer, more preferably in the range of from 45 to 70 nanometer, a Dv50 value in the range of from 45 to 125 nanometer, preferably in the range of from 55 to 115 nanometer, more preferably in the range of from 65 to 105 nanometer, and a Dv90 value in the range of from 65 to 200 nanometer, preferably in the range of from 85 to 180 nanometer, more preferably in the range of from 95 to 160 nanometer, determined as described in Reference Example 5.

It is preferred that the volume-based particle size distribution of the colloidal dispersion of silica is a mono-modal distribution.

As regards the content of the silica in the colloidal dispersion of silica in water, no particular restriction applies. It is preferred that the colloidal dispersion of silica in water comprises the silica in an amount in the range of from 25 to 65 weight-%, more preferably in the range of from 30 to 60 weight-%, more preferably in the range of from 35 to 55 weight-%, based on the total weight of the silica and the water.

It is preferred that from 95 to 100 weight-%, preferably from 98 to 100 weight-%, more preferably from 99 to 100 weight-% of the binder precursor consist of the colloidal dispersion of silica in water.

Further, it is preferred that from 95 to 100 weight-%, preferably from 98 to 100 weight-%, more preferably from 99 to 100 weight-%, more preferably from least 99.5 to 100 weight-%, more preferably from 99.9 to 100 weight-% of the zeolitic material consist of Si, O, Ti and preferably H.

As regards the amount of Ti comprised in the zeolitic material, no particular restriction applies. It is preferred that the zeolitic material comprises Ti in an amount in the range of from 0.2 to 5 weight-%, more preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, based on the total weight of the zeolitic material.

It is preferred that the zeolitic material is titanium silicalite-1.

Further, it is preferred that in the mixture, the weight ratio of the zeolitic material, relative to the sum of the zeolitic material and the binder calculated as SiO₂, is in the range of from 2 to 90%, more preferably in the range of from 5 to 70%, more preferably in the range of from 10 to 50%, more preferably in the range of from 15 to 30%, more preferably in the range of from 20 to 25%.

The mixture may comprise further components. Thus, it is preferred that the mixture further comprises one or more additives, more preferably one or more viscosity modifying agents, or one or more mesopore forming agents, or one or more viscosity modifying agents and one or more mesopore forming agents.

It is preferred that from 95 to 100 weight-%, more preferably from 98 to 100 weight-%, more preferably from 99 to 100 weight-%, more preferably from 99.5 to 100 weight-%, more preferably from 99.9 to 100 weight-% of the mixture consist of the zeolitic material, and the binder precursor. In the case where the mixture further comprises one or more additives, it is preferred that from 95 to 100 weight-%, more preferably from 98 to 100 weight-%, more preferably from 99 to 100 weight-%, more preferably from 99.5 to 100 weight-%, more preferably from 99.9 to 100 weight-% of the mixture consist of the zeolitic material, the binder precursor, and the one or more additives.

It is preferred that the one or more additives are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose ethers, polyalkylene oxides, polystyrenes, and mixtures of two or more thereof, wherein the organic polymers are more preferably selected from the group consisting of methyl celluloses, carboxymethyl celluloses, polyethylene oxides, polystyrenes, and mixtures of two or more thereof, wherein more preferably, the one or more additives comprise, more preferably consist of, water, a carboxymethyl cellulose, a polyethylene oxide, and a polystyrene.

In the case where the one or more additives are selected from the group consisting of water, alcohols, organic polymers, and mixtures of two or more thereof, it is preferred that in the mixture, the weight ratio of the zeolitic material, relative to the one or more additives, is in the range of from 0.3:1 to 1:1, more preferably in the range of from 0.4:1 to 0.8:1, more preferably in the range of from 0.5:1 to 0.6:1.

In the case where the one or more additives comprise a cellulose derivative, preferably a cellulose ether, more preferably a carboxymethyl cellulose, it is preferred that in the mixture, the weight ratio of the zeolitic material, relative to the cellulose derivative, preferably the cellulose ether, more preferably the carboxymethyl cellulose, is in the range of from 10:1 to 53:1, more preferably in the range of from 15:1 to 40:1, more preferably in the range of from 20:1 to 35:1.

In the case where the one or more additives comprise a polyethylene oxide, it is preferred that in the mixture, the weight ratio of the zeolitic material, relative to the polyethylene oxide, is in the range of from 70:1 to 110:1, more preferably in the range of from 75:1 to 100:1, more preferably in the range of from 77:1 to 98:1.

In the case where the one or more additives comprise a polystyrene, it is preferred that in the mixture, the weight ratio of the zeolitic material, relative to the polystyrene, is in the range of from 2:1 to 8:1, more preferably in the range of from 3:1 to 6:1, more preferably in the range of from 3.5:1 to 5:1.

In the case where the one or more additives comprise water, it is preferred that in the mixture, the weight ratio of the zeolitic material, relative to the water, is in the range of from 0.7:1 to 0.85:1, more preferably in the range of from 0.72:1 to 0.8:1, more preferably in the range of from 0.74:1 to 0.0.79:1.

It is particularly preferred that the one or more additives comprise a cellulose derivative, preferably a cellulose ether, more preferably a carboxymethyl cellulose, a polyethylene oxide, a polystyrene, and water.

According to a yet further aspect, the present invention relates to a process for preparing a mixture comprising a zeolitic material, water, and silica, preferably for preparing a mixture as described above, the process comprising
(i′) providing a zeolitic material which exhibits a type I nitrogen adsorption/desorption isotherm, determined as described in Reference Example 1, and which has framework type MFI and a framework structure comprising Si, O, and Ti;
(ii′) providing a colloidal dispersion of silica in water, said silica exhibiting a volume-based particle size distribution characterized by a Dv10 value of at least 35 nanometer, preferably in the range of from 35 to 80 nanometer, more preferably in the range of from 40 to 75 nanometer, more preferably in the range of from 45 to 70 nanometer, a Dv50 value of at least 45 nanometer, preferably in the range of from 45 to 125 nanometer, more preferably in the range of from 55 to 115 nanometer, more preferably in the range of from 65 to 105 nanometer, and a Dv90 value of at least 65 nanometer, preferably in the range of from 65 to 200 nanometer, more preferably in the range of from 85 to 180 nanometer, more preferably in the range of from 95 to 160 nanometer, determined as described in Reference Example 5;
(iii′) preparing a mixture comprising the particles of the zeolitic material provided in (i′) and the binder precursor provided in (ii′).

It is preferred that the volume-based particle size distribution of the colloidal dispersion of silica in water according to (ii′) is characterized by a Dv10 value in the range of from 35 to 80 nanometer, preferably in the range of from 40 to 75 nanometer, more preferably in the range of from 45 to 70 nanometer, a Dv50 value in the range of from 45 to 125 nanometer, preferably in the range of from 55 to 115 nanometer, more preferably in the range of from 65 to 105 nanometer, and a Dv90 value in the range of from 65 to 200 nanometer, preferably in the range of from 85 to 180 nanometer, more preferably in the range of from 95 to 160 nanometer, determined as described in Reference Example 5.

Further, it is preferred that the volume-based particle size distribution of the colloidal dispersion of silica in water according to (ii′) is a mono-modal distribution.

As regards the content of the silica comprised in the colloidal dispersion of silica in water, it is preferred that the colloidal dispersion of silica in water according to (ii′) comprises the silica in an amount in the range of from 25 to 65 weight-%, more preferably in the range of from 30 to 60 weight-%, more preferably in the range of from 35 to 55 weight-%, based on the total weight of the silica and the water.

It is preferred that from 95 to 100 weight-%, more preferably from 98 to 100 weight-%, more preferably from 99 to 100 weight-% of the binder precursor according to (ii′) consist of the colloidal dispersion of silica in water.

Further, it is preferred that from 95 to 100 weight-%, more preferably from 98 to 100 weight-%, more preferably from 99 to 100 weight-%, more preferably from least 99.5 to 100 weight-%, more preferably from 99.9 to 100 weight-% of the zeolitic material according to (i′) consist of Si, O, Ti and preferably H.

As regards the amount of Ti comprised in the zeolitic material according to (i′), no particular restriction applies. It is preferred that the zeolitic material according to (i′) comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, based on the total weight of the zeolitic material.

It is preferred that the zeolitic material according to (i′) is titanium silicalite-1.

As regards the weight ratio of the zeolitic material, relative to the sum of the zeolitic material and the binder calculated as SiO₂, in the mixture prepared according to (iii′). It is preferred that in the mixture prepared according to (iii′), the weight ratio of the zeolitic material, relative to the sum of the zeolitic material and the binder calculated as SiO₂, is in the range of from 2 to 90%, more preferably in the range of from 5 to 70%, more preferably in the range of from 10 to 50%, more preferably in the range of from 15 to 30%, more preferably in the range of from 20 to 25%.

The mixture prepared according to (iii′) may comprise further components. It is preferred that the mixture prepared according to (iii′) further comprises one or more additives, more preferably one or more viscosity modifying agents, or one or more mesopore forming agents, or one or more viscosity modifying agents and one or more mesopore forming agents.

In the case where the mixture prepared according to (iii′) further comprises one or more additives, it is preferred that from 95 to 100 weight-%, more preferably from 98 to 100 weight-%, more preferably from 99 to 100 weight-%, more preferably from 99.5 to 100 weight-%, more preferably from 99.9 to 100 weight-% of the mixture prepared according to (iii′) consist of the zeolitic material, the binder precursor, and the one or more additives.

It is preferred that the one or more additives are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose ethers, polyalkylene oxides, polystyrenes, and mixtures of two or more thereof, wherein the organic polymers are more preferably selected from the group consisting of methyl celluloses, carboxymethyl celluloses, polyethylene oxides, polystyrenes, and mixtures of two or more thereof, wherein more preferably, the one or more additives comprise, more preferably consist of, water, a carboxymethyl cellulose, a polyethylene oxide, and a polystyrene.

In the case where the mixture prepared according to (iii′) comprises one or more additives, it is preferred that in the mixture prepared according to (iii′), the weight ratio of the zeolitic material, relative to the one or more additives, is in the range of from 0.3:1 to 1:1, more preferably in the range of from 0.4:1 to 0.8:1, more preferably in the range of from 0.5:1 to 0.6:1.

In the case where the mixture prepared according to (iii) and subjected to (iv) comprises a cellulose derivative, preferably a cellulose ether, more preferably a carboxymethyl cellulose, it is preferred that in the mixture prepared according to (iii) and subjected to (iv), the weight ratio of the zeolitic material, relative to the cellulose derivative, preferably the cellulose ether, more preferably the carboxymethyl cellulose, is in the range of from 10:1 to 53:1, more preferably in the range of from 15:1 to 40:1, more preferably in the range of from 20:1 to 35:1.

In the case where the mixture prepared according to (iii) and subjected to (iv) comprises a polyethylene oxide, it is preferred that in the mixture prepared according to (iii) and subjected to (iv), the weight ratio of the zeolitic material, relative to the polyethylene oxide, is in the range of from 70:1 to 110:1, more preferably in the range of from 75:1 to 100:1, more preferably in the range of from 77:1 to 98:1;

In the case where the mixture prepared according to (iii) and subjected to (iv) comprises water, it is preferred that in the mixture prepared according to (iii) and subjected to (iv), the weight ratio of the zeolitic material, relative to the water, is in the range of from 0.7:1 to 0.85:1, more preferably in the range of from 0.72:1 to 0.8:1, more preferably in the range of from 0.74:1 to 0.0.79:1.

It is preferred that preparing the mixture according to (iii) comprises mixing in a kneader or in a mix-muller.

Further, it is preferred that the process for preparing a mixture comprising a zeolitic material, water, and silica, as described herein consists of steps (i), (ii) and (iii).

According to a yet further aspect of the present invention, the present invention relates to a mixture, preferably the mixture as described herein, obtainable or obtained by a process as described herein.

The present invention is further illustrated by the following set of embodiments and combinations of embodiments resulting from the dependencies and back-references as indicated. In particular, it is noted that in each instance where a range of embodiments is mentioned, for example in the context of a term such as “The molding of any one of embodiments 1 to 4”, every embodiment in this range is meant to be explicitly disclosed for the skilled person, i.e. the wording of this term is to be understood by the skilled person as being synonymous to “The molding of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A chemical molding comprising a zeolitic material which exhibits a type I nitrogen adsorption/desorption isotherm determined as described in Reference Example 1, and which has framework type MFI and a framework structure comprising Si, O, and Ti, the molding further comprising a binder for said zeolitic material, the binder comprising Si and O, wherein the molding exhibits a total pore volume of at least 0.4 mL/g, determined as described in Reference Example 2, and a crushing strength of at least 6 N, determined as described in Reference Example 3.
2. The molding of embodiment 1, wherein from 95 to 100 weight-%, preferably from 98 to 100 weight-%, more preferably from 99 to 100 weight-%, more preferably from least 99.5 to 100 weight-%, more preferably from 99.9 to 100 weight-% of the zeolitic material comprised in the molding consist of Si, O, Ti and optionally H.
3. The molding of embodiment 1 or 2, wherein the zeolitic material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the zeolitic material.
4. The molding of any one of embodiments 1 to 3, wherein the zeolitic material comprised in the molding is titanium silicalite-1.
5. The molding of any one of embodiments 1 to 4, wherein from 95 to 100 weight-%, preferably from 98 to 100 weight-%, more preferably from 99 to 100 weight-%, more preferably from at least 99.5 to 100 weight-%, more preferably from 99.9 to 100 weight-% of the binder comprised in the molding consist of Si and O.
6. The molding of any one of embodiments 1 to 5, comprising the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the molding.
7. The molding of any one of embodiments 1 to 6, wherein from 95 to 100 weight-%, preferably from 98 to 100 weight-%, more preferably from 99 to 100 weight-%, more preferably from least 99.5 to 100 weight-%, more preferably from 99.9 to 100 weight-% of the molding consist of the zeolitic material and the binder.
8. The molding of any one of embodiments 1 to 7, comprising micropores having a pore diameter in the range of from 0.1 to less than 2 nm, determined as described in Reference Example 4, and mesopores having a pore diameter in the range of from 2 to 50 nm, determined as described in Reference Example 4.
9. The molding of any one of embodiments 1 to 8, exhibiting a total pore volume in the range of from 0.4 to 1.5 mL/g, preferably in the range of from 0.4 to 1.2 mL/g, more preferably in the range of from 0.4 to 1.0 mL/g.
10. The molding of any one of embodiments 1 to 9, exhibiting a crushing strength in the range of from 6 to 25 N, preferably in the range of from 7 to 20 N, more preferably in the range of from 8 to 15 N.
11. The molding of any one of embodiments 1 to 10, being a strand, preferably having a hexagonal, rectangular, quadratic, triangular, oval, or circular cross-section, more preferably a circular cross-section.
12. The molding of embodiment 11, wherein the cross-section has a diameter in the range of from 0.5 to 5 mm, preferably in the range of from 1 to 3 mm, more preferably in the range of from 1.5 to 2 mm.
13. The molding of any one of embodiments 1 to 12, preferably 11 or 12, being an extrudate.
14. The molding of any one of embodiments 1 to 13, exhibiting a tortuosity parameter relative to water in the range of from 1.0 to 2.5, preferably in the range of from 1.3 to 2.0, more preferably in the range of from 1.6 to 1.8, more preferably in the range of from 1.6 to 1.75, more preferably in the range of from 1.6 to 1.72, determined as described in Reference Example 11.
15. The molding of any one of embodiments 1 to 14, exhibiting a BET specific surface area in the range of from 300 to 450 m²/g, preferably in the range of from 310 to 400 m²/g, more preferably in the range of from 320 to 375 m²/g, determined as described in Reference Example 6.
16. The molding of any one of embodiments 1 to 15, exhibiting a crystallinity in the range of from 50 to 100%, preferably in the range of from 50 to 90%, more preferably in the range of from 50 to 80%, determined as described in Reference Example 7.
17. The molding of any one of embodiments 1 to 16, exhibiting a propylene oxide activity of at least 4.5 weight-%, preferably in the range of from 4.5 to 11 weight-%, more preferably in the range of from 4.5 to 10 weight-%, determined as described in Reference Example 9.
18. The molding of any one of embodiments 1 to 17, exhibiting a pressure drop rate in the range of from 0.005 to 0.019 bar(abs)/min, preferably in the range of from 0.006 to 0.017 bar(abs)/min, more preferably in the range of from 0.007 to 0.015 bar(abs)/min, determined as described in Reference Example 9.
19. The molding of any one of embodiments 1 to 18, used as catalyst in a reaction for preparing propylene oxide from propene and hydrogen peroxide, wherein the catalyst exhibits a hydrogen peroxide conversion in the range of from 90 to 95%, determined in a continuous epoxidation reaction as described in Reference Example 10 at a temperature of the cooling medium in the range of from 55 to 56° C. at a time on stream in the range of from 200 to 600 hours, preferably at a time on stream in the range of from 300 to 600 hours, more preferably at a time on stream in the range of from 350 to 600 hours, wherein the term “time on stream” refers to the duration of the continuous epoxidation reaction without regeneration of the catalyst.
20. A process for preparing a chemical molding comprising a zeolitic material which exhibits a type I nitrogen adsorption/desorption isotherm determined as described in Reference Example 1, and which has framework type MFI and a framework structure comprising Si, O, and Ti, the molding further comprising a binder for said zeolitic material, the binder comprising Si and O, preferably for preparing a chemical molding according to any one of embodiments 1 to 19, the process comprising
   (i) providing a zeolitic material exhibiting a type I nitrogen adsorption/desorption isotherm determined as described in Reference Example 1, having framework type MFI and a framework structure comprising Si, O, and Ti;
   (ii) providing a binder precursor comprising a colloidal dispersion of silica in water, said binder precursor exhibiting a volume-based particle size distribution characterized by a Dv10 value of at least 35 nanometer, a Dv50 value of at least 45 nanometer, and a Dv90 value of at least 65 nanometer, determined as described in Reference Example 5;
   (iii) preparing a mixture comprising the zeolitic material provided in (i) and the binder precursor provided in (ii);
   (iv) shaping the mixture obtained from (iii), obtaining a precursor of the molding;
   (v) preparing a mixture comprising the precursor of the molding obtained from (iv) and water, and subjecting the mixture to a water treatment under hydrothermal conditions, obtaining a water-treated precursor of the molding;
   (vi) calcining the water-treated precursor of the molding in a gas atmosphere, obtaining the molding.
21. The process of embodiment 20, wherein the volume-based particle size distribution of the colloidal dispersion of silica in water according to (ii) is characterized by a Dv10 value in the range of from 35 to 80 nanometer, preferably in the range of from 40 to 75 nanometer, more preferably in the range of from 45 to 70 nanometer, a Dv50 value in the range of from 45 to 125 nanometer, preferably in the range of from 55 to 115 nanometer, more preferably in the range of from 65 to 105 nanometer, and a Dv90 value in the range of from 65 to 200 nanometer, preferably in the range of from 85 to 180 nanometer, more preferably in the range of from 95 to 160 nanometer, determined as described in Reference Example 5.
22. The process of embodiment 20 or 21, wherein the volume-based particle size distribution of the colloidal dispersion of silica in water according to (ii) is a mono-modal distribution.
23. The process of any one of embodiments 20 to 22, wherein the colloidal dispersion of silica in water according to (ii) comprises the silica in an amount in the range of from 25 to 65 weight-%, preferably in the range of from 30 to 60 weight-%, more preferably in the range of from 35 to 55 weight-%, based on the total weight of the silica and the water.
24. The process of any one of embodiments 20 to 23, wherein from 95 to 100 weight-%, preferably from 98 to 100 weight-%, more preferably from 99 to 100 weight-% of the binder precursor according to (ii) consist of the colloidal dispersion of silica in water.
25. The process of any one of embodiments 20 to 24, wherein from 95 to 100 weight-%, preferably from 98 to 100 weight-%, more preferably from 99 to 100 weight-%, more preferably from least 99.5 to 100 weight-%, more preferably from 99.9 to 100 weight-% of the zeolitic material according to (i) consist of Si, O, Ti and preferably H.
26. The process of any one of embodiments 20 to 25, wherein the zeolitic material according to (i) comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, based on the total weight of the zeolitic material.
27. The process of any one of embodiments 20 to 26, wherein the zeolitic material according to (i) is titanium silicalite-1.
28. The process of any one of embodiments 20 to 27, wherein in the mixture prepared according to (iii) and subjected to (iv), the weight ratio of the zeolitic material, relative to the sum of the zeolitic material and the binder calculated as SiO₂, is in the range of from 2 to 90%, preferably in the range of from 5 to 70%, more preferably in the range of from 10 to 50%, more preferably in the range of from 15 to 30%, more preferably in the range of from 20 to 25%.
29. The process of any one of embodiments 20 to 28, wherein the mixture prepared according to (iii) and subjected to (iv) further comprises one or more additives, preferably one or more viscosity modifying agents, or one or more mesopore forming agents, or one or more viscosity modifying agents and one or more mesopore forming agents.
30. The process of embodiment 29, wherein from 95 to 100 weight-%, preferably from 98 to 100 weight-%, more preferably from 99 to 100 weight-%, more preferably from 99.5 to 100 weight-%, more preferably from 99.9 to 100 weight-% of the mixture prepared according to (iii) and subjected to (iv) consist of the zeolitic material, the binder precursor, and the one or more additives.
31. The process of embodiment 29 or 30, wherein the one or more additives are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose ethers, polyalkylene oxides, polystyrenes, and mixtures of two or more thereof, wherein the organic polymers are more preferably selected from the group consisting of a methyl celluloses, carboxymethyl celluloses, polyethylene oxides, polystyrenes, and mixtures of two or more thereof, wherein more preferably, the one or more additives comprise, more preferably consist of, water, a carboxymethyl cellulose, a polyethylene oxide, and a polystyrene.
32. The process of any one of embodiments 29 to 31, wherein in the mixture prepared according to (iii) and subjected to (iv), the weight ratio of the zeolitic material, relative to the one or more additives, is in the range of from 0.3:1 to 1:1, preferably in the range of from 0.4:1 to 0.8:1, more preferably in the range of from 0.5:1 to 0.6:1.
33. The process of any one of embodiment 29 to 32, wherein in the mixture prepared according to (iii) and subjected to (iv),
the weight ratio of the zeolitic material, relative to the cellulose derivative, preferably the cellulose ether, more preferably the carboxymethyl cellulose, is in the range of from 10:1 to 53:1, preferably in the range of from 15:1 to 40:1, more preferably in the range of from 20:1 to 35:1;
the weight ratio of the zeolitic material, relative to the polyethylene oxide, is in the range of from 70:1 to 110:1, preferably in the range of from 75:1 to 100:1, more preferably in the range of from 77:1 to 98:1;
the weight ratio of the zeolitic material, relative to the polystyrene, is in the range of from 2:1 to 8:1, preferably in the range of from 3:1 to 6:1, more preferably in the range of from 3.5:1 to 5:1;
the weight ratio of the zeolitic material, relative to the water, is in the range of from 0.7:1 to 0.85:1, preferably in the range of from 0.72:1 to 0.8:1, more preferably in the range of from 0.74:1 to 0.0.79:1.
34. The process of any one of embodiments 20 to 33, wherein preparing the mixture according to (iii) comprises mixing in a kneader or in a mix-muller.
35. The process of any one of embodiments 20 to 34, wherein according to (iv), the mixture obtained from (iii) is shaped to a strand, preferably to a strand having a circular cross-section, wherein the strand having a circular cross-section has a diameter preferably in the range of from 0.5 to 5 mm, more preferably in the range of from 1 to 3 mm, more preferably in the range of from 1.5 to 2 mm.
36. The process of any one of embodiments 20 to 35, wherein the mixture obtained from (iii) and subjected to (iv) has a plasticity in the range of from 500 to 3000 N, preferably in the range of from 750 to 2000 N, more preferably in the range of from 1000 to 1500 N, determined as described in Reference Example 12.
37. The process of any one of embodiments 20 to 36, wherein shaping according to (iv) comprises extruding the mixture obtained from (iii).
38. The process of any one of embodiments 20 to 37, wherein shaping according to (iv) further comprises drying the precursor of the molding in a gas atmosphere, wherein said drying is preferably carried out at a temperature of the gas atmosphere in the range of from 80 to 160° C., preferably in the range of from 100 to 140° C., more preferably in the range of from 110 to 130° C., wherein the gas atmosphere preferably comprises nitrogen, oxygen, or a mixture thereof, wherein the gas atmosphere is more preferably oxygen, air, or lean air.
39. The process of any one of embodiments 20 to 38, preferably of embodiment 38, wherein shaping according to (iv) further comprises calcining the preferably dried precursor of the molding in a gas atmosphere, wherein calcining is preferably carried out at a temperature of the gas atmosphere in the range of from 450 to 530° C., preferably in the range of from 470 to 510° C., more preferably in the range of from 480 to 500° C., wherein the gas atmosphere comprises preferably nitrogen, oxygen, or a mixture thereof, wherein the gas atmosphere is more preferably oxygen, air, or lean air.
40. The process of any one of embodiments 20 to 39, wherein in the mixture prepared in (v), the weight ratio of the precursor of the molding relative to the water is in the range of from 1:1 to 1:30, preferably in the range of from 1:5 to 1:25, more preferably in the range of from 1:10 to 1:20.
41. The process of any one of embodiments 20 to 40, wherein from 95 to 100 weight-%, preferably from 98 to 100 weight-%, more preferably from 99 to 100 weight-%, more preferably from 99.5 to 100 weight-%, more preferably from 99.9 to 100 weight-% of the mixture prepared according to (v) consist of the precursor of the molding and water.
42. The process of any one of embodiments 20 to 41, wherein the water treatment according to (v) comprises a temperature of the mixture in the range of from 100 to 200° C., preferably in the range of from 125 to 175° C., more preferably in the range of from 130 to 160° C., more preferably in the range of from 135 to 155° C. more preferably in the range of from 140 to 150° C.
43. The process of any one of embodiments 20 to 42, wherein the water treatment according to (v) is carried out under autogenous pressure, preferably in an autoclave.
44. The process of any one of embodiments 20 to 43, wherein the water treatment according to (v) is carried out for 6 to 10 h, preferably for 7 to 9 h, more preferably for 7.5 to 8.5 h.
45. The process of any one of embodiments 20 to 44, wherein (v) further comprises separating the water-treated precursor of the molding from the mixture obtained from the water treatment.
46. The process of embodiment 45, wherein separating the water-treated precursor of the molding from the mixture obtained from the water treatment comprises subjecting the mixture obtained from the water treatment to solid-liquid separation, preferably washing the separated precursor, and preferably drying the preferably washed precursor.
47. The process of embodiment 46, wherein the solid-liquid separation according to (v) comprises filtration, or centrifugation, or filtration and centrifugation.
48. The process of embodiment 46 or 47, wherein washing according to (v) comprises washing the precursor at least once with a liquid solvent system, wherein the liquid solvent system preferably comprises one or more of water, an alcohol, and a mixture of two or more thereof, wherein the water-treated precursor of the molding is more preferably washed with water.
49. The process of any one of embodiments 46 to 48, wherein drying according to (v) comprises drying the precursor in a gas atmosphere, wherein drying is preferably carried out at a temperature of the gas atmosphere in the range of from 80 to 160° C., more preferably in the range of from 100 to 140° C., more preferably in the range of from 110 to 130° C., wherein the gas atmosphere preferably comprises nitrogen, oxygen, or a mixture thereof, wherein the gas atmosphere is more preferably oxygen, air or lean air.
50. The process of any one of embodiments 20 to 49, wherein calcining according to (vi) is carried out at a temperature of the gas atmosphere in the range of from 400 to 490° C., preferably in the range of from 420 to 470° C., more preferably in the range of from 440 to 460° C., wherein the gas atmosphere preferably comprises nitrogen, oxygen, or a mixture thereof, wherein the gas atmosphere is more preferably oxygen, air or lean air.
51. The process of any one of embodiments 20 to 50, consisting of (i), (ii), (iii), (iv), (v) and (vi).
52. A chemical molding comprising particles of a zeolitic material exhibiting a type I nitrogen adsorption/desorption isotherm determined as described in Reference Example 1, having framework type MFI and a framework structure comprising Si, O, and Ti, the molding further comprising a binder for said particles, the binder comprising Si and O, preferably the chemical molding according to any one of embodiments 1 to 19, obtainable or obtained by a process according to any one of embodiments 20 to 51.
53. Use of a molding according to any one of embodiments 1 to 19 or according to embodiment 52 as an adsorbent, an absorbent, a catalyst or a catalyst component, preferably as a catalyst or as a catalyst component, more preferably as a Lewis acid catalyst or a Lewis acid catalyst component, as an isomerization catalyst or as an isomerization catalyst component, as an oxidation catalyst or as an oxidation catalyst component, as an aldol condensation catalyst or as an aldol condensation catalyst component, or as a Prins reaction catalyst or as a Prins reaction catalyst component.
54. The use of embodiment 53 as an oxidation catalyst or as an oxidation catalyst component, preferably as an epoxidation catalyst or as an epoxidation catalyst component, more preferably as an epoxidation catalyst.
55. The use of embodiment 54 for the epoxidation reaction of an organic compound having at least one C—C double bond, preferably a C2-C10 alkene, more preferably a C2-C5 alkene, more preferably a C2-C4 alkene, more preferably a C2 or C3 alkene, more preferably propene, more preferably for the epoxidation of propene with hydrogen peroxide as oxidizing agent, more preferably for the epoxidation of propene with hydrogen peroxide as oxidizing agent in a solvent comprising an alcohol, preferably methanol.
56. A process for oxidizing an organic compound comprising bringing the organic compound in contact, preferably in continuous mode, with a catalyst comprising a molding according to any one of embodiments 1 to 19 or according to embodiment 52, preferably for epoxidizing an organic compound, more preferably for epoxidizing an organic compound having at least one C—C double bond, preferably a C2-C10 alkene, more preferably a C2-C5 alkene, more preferably a C2-C4 alkene, more preferably a C2 or C3 alkene, more preferably propene.
57. The process of embodiment 56, wherein hydrogen peroxide is used as oxidizing agent, wherein the oxidation reaction is preferably carried out in a solvent, more preferably in a solvent comprising an alcohol, preferably methanol.
58. A process for preparing propylene oxide comprising reacting propene, preferably in continuous mode, with hydrogen peroxide in methanolic solution in the presence of a catalyst comprising a molding according to any one of embodiments 1 to 19 or according to embodiment 52 to obtain propylene oxide.
59. Use of a colloidal dispersion of silica in water as a binder precursor for preparing a chemical molding comprising a zeolitic material which exhibits a type I nitrogen adsorption/desorption isotherm determined as described in Reference Example 1, and which has framework type MFI and a framework structure comprising Si, O, and Ti, the molding further comprising a binder resulting from said binder precursor, preferably for preparing a molding according to any one of embodiments 1 to 19, said silica exhibiting a volume-based particle size distribution characterized by a Dv10 value of at least 35 nanometer, preferably in the range of from 35 to 80 nanometer, more preferably in the range of from 40 to 75 nanometer, more preferably in the range of from 45 to 70 nanometer, a Dv50 value of at least 45 nanometer, preferably in the range of from 45 to 125 nanometer, more preferably in the range of from 55 to 115 nanometer, more preferably in the range of from 65 to 105 nanometer, and a Dv90 value of at least 65 nanometer, preferably in the range of from 65 to 200 nanometer, more preferably in the range of from 85 to 180 nanometer, more preferably in the range of from 95 to 160 nanometer, determined as described in Reference Example 5, said molding preferably exhibiting a total pore volume of at least 0.4 mL/g, determined as described in Reference Example 2, and a crushing strength of at least 6 N, determined as described in Reference Example 3.

The present invention is further illustrated by the further following set of embodiments and combinations of embodiments resulting from the dependencies and back-references as indicated. In particular, it is noted that in each instance where a range of embodiments is mentioned, for example in the context of a term such as “The mixture of any one of embodiments 1′ to 4′”, every embodiment in this range is meant to be explicitly disclosed for the skilled person, i.e. the wording of this term is to be understood by the skilled person as being synonymous to “The mixture of any one of embodiments 1′, 2′, 3′, and 4′”. Further, it is explicitly noted that the following set of embodiments is not the set of claims determining the extent of protection, but represents a suitably structured part of the description directed to general and preferred aspects of the present invention.
1′. A mixture comprising a zeolitic material which exhibits a type I nitrogen adsorption/desorption isotherm determined as described in Reference Example 1, and which has framework type MFI and a framework structure comprising Si, O, and Ti, the mixture further comprising a colloidal dispersion of silica in water, said binder precursor exhibiting a volume-based particle size distribution characterized by a Dv10 value of at least 35 nanometer, a Dv50 value of at least 45 nanometer, and a Dv90 value of at least 65 nanometer, determined as described in Reference Example 5.
2′. The mixture of embodiment 1′, having a plasticity in the range of from 500 to 3000 N, preferably in the range of from 750 to 2000 N, more preferably in the range of from 1000 to 1500 N, determined as described in Reference Example 12.
3′. The mixture of embodiment 1′ or 2′, wherein the volume-based particle size distribution of the colloidal dispersion of silica in water is characterized by a Dv10 value in the range of from 35 to 80 nanometer, preferably in the range of from 40 to 75 nanometer, more preferably in the range of from 45 to 70 nanometer, a Dv50 value in the range of from 45 to 125 nanometer, preferably in the range of from 55 to 115 nanometer, more preferably in the range of from 65 to 105 nanometer, and a Dv90 value in the range of from 65 to 200 nanometer, preferably in the range of from 85 to 180 nanometer, more preferably in the range of from 95 to 160 nanometer, determined as described in Reference Example 5.
4′. The mixture of any one of embodiments 1′ to 3′, wherein the volume-based particle size distribution of the colloidal dispersion of silica is a mono-modal distribution.
5′. The mixture of any one of embodiments 1′ to 4′, wherein the colloidal dispersion of silica in water comprises the silica in an amount in the range of from 25 to 65 weight-%, preferably in the range of from 30 to 60 weight-%, more preferably in the range of from 35 to 55 weight-%, based on the total weight of the silica and the water.
6′. The mixture of any one of embodiments 1′ to 5′, wherein from 95 to 100 weight-%, preferably from 98 to 100 weight-%, more preferably from 99 to 100 weight-% of the binder precursor consist of the colloidal dispersion of silica in water.
7′. The mixture of any one of embodiments 1′ to 6′, wherein from 95 to 100 weight-%, preferably from 98 to 100 weight-%, more preferably from 99 to 100 weight-%, more preferably from least 99.5 to 100 weight-%, more preferably from 99.9 to 100 weight-% of the zeolitic material consist of Si, O, Ti and preferably H.
8′. The mixture of any one of embodiments 1′ to 7′, wherein the zeolitic material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, based on the total weight of the zeolitic material, wherein the zeolitic material is preferably titanium silicalite-1.
9′. The mixture of any one of embodiments 1′ to 8′, wherein in the mixture, the weight ratio of the zeolitic material, relative to the sum of the zeolitic material and the binder calculated as SiO₂, is in the range of from 2 to 90%, preferably in the range of from 5 to 70%, more preferably in the range of from 10 to 50%, more preferably in the range of from 15 to 30%, more preferably in the range of from 20 to 25%.
10′. The mixture of any one of embodiments 1′ to 9′, further comprising one or more additives, preferably one or more viscosity modifying agents, or one or more mesopore forming agents, or one or more viscosity modifying agents and one or more mesopore forming agents.
11′. The mixture of any one of embodiments 1′ to 10′, wherein from 95 to 100 weight-%, preferably from 98 to 100 weight-%, more preferably from 99 to 100 weight-%, more preferably from 99.5 to 100 weight-%, more preferably from 99.9 to 100 weight-% of the mixture consist of the zeolitic material, the binder precursor, and the one or more additives.
12′. The mixture of any one of embodiments 1′ to 11′, wherein the one or more additives are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose ethers, polyalkylene oxides, polystyrenes, and mixtures of two or more thereof, wherein the organic polymers are more preferably selected from the group consisting of methyl celluloses, carboxymethyl celluloses, polyethylene oxides, polystyrenes, and mixtures of two or more thereof, wherein more preferably, the one or more additives comprise, more preferably consist of, water, a carboxymethyl cellulose, a polyethylene oxide, and a polystyrene.
13′. The mixture of embodiment 12′, wherein in the mixture, the weight ratio of the zeolitic material, relative to the one or more additives, is in the range of from 0.3:1 to 1:1, preferably in the range of from 0.4:1 to 0.8:1, more preferably in the range of from 0.5:1 to 0.6:1.
14′. The mixture of embodiment 12′ or 13′, wherein in the mixture,
the weight ratio of the zeolitic material, relative to the cellulose derivative, preferably the cellulose ether, more preferably the carboxymethyl cellulose, is in the range of from 10:1 to 53:1, preferably in the range of from 15:1 to 40:1, more preferably in the range of from 20:1 to 35:1;
the weight ratio of the zeolitic material, relative to the polyethylene oxide, is in the range of from 70:1 to 110:1, preferably in the range of from 75:1 to 100:1, more preferably in the range of from 77:1 to 98:1;
the weight ratio of the zeolitic material, relative to the polystyrene, is in the range of from 2:1 to 8:1, preferably in the range of from 3:1 to 6:1, more preferably in the range of from 3.5:1 to 5:1;
the weight ratio of the zeolitic material, relative to the water, is in the range of from 0.7:1 to 0.85:1, preferably in the range of from 0.72:1 to 0.8:1, more preferably in the range of from 0.74:1 to 0.0.79:1.
15′. A process for preparing a mixture comprising a zeolitic material, water, and silica, preferably for preparing a mixture according to any one of embodiments 1′ to 14′, the process comprising
   (i′) providing a zeolitic material which exhibits a type I nitrogen adsorption/desorption isotherm determined as described in Reference Example 1, and which has framework type MFI and a framework structure comprising Si, O, and Ti;
   (ii′) providing a colloidal dispersion of silica in water, said silica exhibiting a volume-based particle size distribution characterized by a Dv10 value of at least 35 nanometer, preferably in the range of from 35 to 80 nanometer, more preferably in the range of from 40 to 75 nanometer, more preferably in the range of from 45 to 70 nanometer, a Dv50 value of at least 45 nanometer, preferably in the range of from 45 to 125 nanometer, more preferably in the range of from 55 to 115 nanometer, more preferably in the range of from 65 to 105 nanometer, and a Dv90 value of at least 65 nanometer, preferably in the range of from 65 to 200 nanometer, more preferably in the range of from 85 to 180 nanometer, more preferably in the range of from 95 to 160 nanometer, determined as described in Reference Example 5;
   (iii′) preparing a mixture comprising the particles of the zeolitic material provided in (i′) and the binder precursor provided in (ii′).
16′. The process of embodiment 15′, wherein the volume-based particle size distribution of the colloidal dispersion of silica in water according to (ii′) is characterized by a Dv10 value in the range of from 35 to 80 nanometer, preferably in the range of from 40 to 75 nanometer, more preferably in the range of from 45 to 70 nanometer, a Dv50 value in the range of from 45 to 125 nanometer, preferably in the range of from 55 to 115 nanometer, more preferably in the range of from 65 to 105 nanometer, and a Dv90 value in the range of from 65 to 200 nanometer, preferably in the range of from 85 to 180 nanometer, more preferably in the range of from 95 to 160 nanometer, determined as described in Reference Example 5.
17′. The process of embodiment 15′ or 16′, wherein the volume-based particle size distribution of the colloidal dispersion of silica in water according to (ii′) is a mono-modal distribution.
18′. The process of any one of embodiments 15′ to 17′, wherein the colloidal dispersion of silica in water according to (ii′) comprises the silica in an amount in the range of from 25 to 65 weight-%, preferably in the range of from 30 to 60 weight-%, more preferably in the range of from 35 to 55 weight-%, based on the total weight of the silica and the water.
19′. The process of any one of embodiments 15′ to 18′, wherein from 95 to 100 weight-%, preferably from 98 to 100 weight-%, more preferably from 99 to 100 weight-% of the binder precursor according to (ii′) consist of the colloidal dispersion of silica in water.
20′. The process of any one of embodiments 15′ to 19′, wherein from 95 to 100 weight-%, preferably from 98 to 100 weight-%, more preferably from 99 to 100 weight-%, more preferably from least 99.5 to 100 weight-%, more preferably from 99.9 to 100 weight-% of the zeolitic material according to (i′) consist of Si, O, Ti and preferably H.
21′. The process of any one of embodiments 15′ to 20′, wherein the zeolitic material according to (i′) comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, based on the total weight of the zeolitic material.
22′. The process of any one of embodiments 15′ to 21′, wherein the zeolitic material according to (i′) is titanium silicalite-1.
23′. The process of any one of embodiments 15′ to 22′, wherein in the mixture prepared according to (iii′), the weight ratio of the zeolitic material, relative to the sum of the zeolitic material and the binder calculated as SiO₂, is in the range of from 2 to 90%, preferably in the range of from 5 to 70%, more preferably in the range of from 10 to 50%, more preferably in the range of from 15 to 30%, more preferably in the range of from 20 to 25%.
24′. The process of any one of embodiments 15′ to 23′, wherein the mixture prepared according to (iii′) further comprises one or more additives, preferably one or more viscosity modifying agents, or one or more mesopore forming agents, or one or more viscosity modifying agents and one or more mesopore forming agents.
25′. The process of embodiment 24′, wherein from 95 to 100 weight-%, preferably from 98 to 100 weight-%, more preferably from 99 to 100 weight-%, more preferably from 99.5 to 100 weight-%, more preferably from 99.9 to 100 weight-% of the mixture prepared according to (iii′) consist of the zeolitic material, the binder precursor, and the one or more additives.
26′. The process of embodiment 24′ or 25′, wherein the one or more additives are selected from the group consisting of water, alcohols, organic polymers, and mixtures of two or more thereof, wherein the organic polymers are preferably selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more preferably selected from the group consisting of cellulose ethers, polyalkylene oxides, polystyrenes, and mixtures of two or more thereof, wherein the organic polymers are more preferably selected from the group consisting of a methyl celluloses, carboxymethyl celluloses, polyethylene oxides, polystyrenes, and mixtures of two or more thereof, wherein more preferably, the one or more additives comprise, more preferably consist of, water, a carboxymethyl cellulose, a polyethylene oxide, and a polystyrene.
27′. The process of any one of embodiments 24′ to 26′, wherein in the mixture prepared according to (iii′), the weight ratio of the zeolitic material, relative to the one or more additives, is in the range of from 0.3:1 to 1:1, preferably in the range of from 0.4:1 to 0.8:1, more preferably in the range of from 0.5:1 to 0.6:1.
28′. The process of any one of embodiment 24′ to 27′, wherein in the mixture prepared according to (iii) and subjected to (iv),
the weight ratio of the zeolitic material, relative to the cellulose derivative, preferably the cellulose ether, more preferably the carboxymethyl cellulose, is in the range of from 10:1 to 53:1, preferably in the range of from 15:1 to 40:1, more preferably in the range of from 20:1 to 0.5:1;
the weight ratio of the zeolitic material, relative to the polyethylene oxide, is in the range of from 70:1 to 110:1, preferably in the range of from 75:1 to 100:1, more preferably in the range of from 77:1 to 98:1;
the weight ratio of the zeolitic material, relative to the polystyrene, is in the range of from 2:1 to 8:1, preferably in the range of from 3:1 to 6:1, more preferably in the range of from 3.5:1 to 5:1;
the weight ratio of the zeolitic material, relative to the water, is in the range of from 0.7:1 to 0.85:1, preferably in the range of from 0.72:1 to 0.8:1, more preferably in the range of from 0.74:1 to 0.0.79:1.
29′. The process of any one of embodiments 15′ to 28′, wherein preparing the mixture according to (iii) comprises mixing in a kneader or in a mix-muller.
30′. The process of any one of embodiments 15′ to 29′, consisting of steps (i), (ii) and (iii).
31′. A mixture, preferably the mixture of any one of embodiments 1′ to 14′, obtainable or obtained by a process according to any one of embodiments 15′ to 29′.
32′. Use of the mixture according to any one of embodiments 1′ to 14′ or according to embodiment 31′ for preparing a chemical molding, preferably a chemical molding according to any one of embodiments 1 to 19 or according to embodiment 52.

The present invention is further illustrated by the following Reference Examples, Examples, and Comparative Examples.

### REFERENCE EXAMPLE 1: DETERMINATION OF N₂ ADSORPTION/DESORPTION ISOTHERMS

The nitrogen adsorption/desorption isotherms were determined at 77 K according to the method disclosed in DIN 66131. The isotherms, at the temperature of liquid nitrogen, were measured using Micrometrics ASAP 2020M and Tristar system.

### REFERENCE EXAMPLE 2: DETERMINATION OF THE TOTAL PORE VOLUME

The total pore volume was determined via intrusion mercury porosimetry according to DIN 66133.

### REFERENCE EXAMPLE 3: DETERMINATION OF THE CRUSHING STRENGTH

The crush strength as referred to in the context of the present invention is to be understood as having been determined via a crush strength test machine Z2.5/TS1S, supplier Zwick GmbH & Co., D-89079 Ulm, Germany. As to fundamentals of this machine and its operation, reference is made to the respective instructions handbook “Register 1: Betriebsanleitung/Sicherheitshandbuch für die Material-Prüfmaschine Z2.5/TS1S ”, version 1.5, December 2001 by Zwick GmbH & Co. Technische Dokumentation, August-Nagel-Strasse 11, D-89079 Ulm, Germany. The machine was equipped with a fixed horizontal table on which the strand was positioned. A plunger having a diameter of 3 mm which was freely movable in vertical direction actuated the strand against the fixed table. The apparatus was operated with a preliminary force of 0.5 N, a shear rate under preliminary force of 10 mm/min and a subsequent testing rate of 1.6 mm/min. The vertically movable plunger was connected to a load cell for force pick-up and, during the measurement, moved toward the fixed turntable on which the molding (strand) to be investigated is positioned, thus actuating the strand against the table. The plunger was applied to the strands perpendicularly to their longitudinal axis. With said machine, a given strand as described below was subjected to an increasing force via a plunger until the strand was crushed. The force at which the strand crushes is referred to as the crushing strength of the strand. Controlling the experiment was carried out by means of a computer which registered and evaluated the results of the measurements. The values obtained are the mean value of the measurements for 10 strands in each case.

### REFERENCE EXAMPLE 5: DETERMINATION OF Dv10, Dv50, AND Dv90 VALUES

The samples were analysed with Zetasizer Nano from Malvern Instruments GmbH, Herrenberg, Germany. First, the pH values of a given sample was determined in order to allow a dilution in the same pH range. The samples were diluted with Millipore water, pH=9.1, to a measurement concentration of 0.005% and then filtrated (5 micrometer). The measurement was carried out atg 25° C.

### REFERENCE EXAMPLE 6: DETERMINATION OF THE BET SPECIFIC SURFACE AREA

The BET specific surface area was determined via nitrogen physisorption at 77 K according to the method disclosed in DIN 66131. The N₂ sorption isotherms at the temperature of liquid nitrogen were measured using Micrometrics ASAP 2020M and Tristar system for determining the BET specific surface area.

### REFERENCE EXAMPLE 7: X-RAY POWDER DIFFRACTION AND DETERMINATION OF THE CRYSTALLINITY

Powder X-ray diffraction (PXRD) data was collected using a diffractometer (D8 Advance Series II, Bruker AXS GmbH) equipped with a LYNXEYE detector operated with a Copper anode X-ray tube running at 40 kV and 40 mA. The geometry was Bragg-Brentano, and air scattering was reduced using an air scatter shield.

Computing crystallinity: The crystallinity of the samples was determined using the software DIFFRAC.EVA provided by Bruker AXS GmbH, Karlsruhe. The method is described on page 121 of the user manual. The default parameters for the calculation were used.

Computing phase composition: The phase composition was computed against the raw data using the modelling software DIFFRAC.TOPAS provided by Bruker AXS GmbH, Karlsruhe. The crystal structures of the identified phases, instrumental parameters as well the crystallite size of the individual phases were used to simulate the diffraction pattern. This was fit against the data in addition to a function modelling the background intensities.

Data collection: The samples were homogenized in a mortar and then pressed into a standard flat sample holder provided by Bruker AXS GmbH for Bragg-Brentano geometry data collection. The flat surface was achieved using a glass plate to compress and flatten the sample powder. The data was collected from the angular range 2 to 70° 2Theta with a step size of 0.02° 2Theta, while the variable divergence slit was set to an angle of 0.1°. The crystalline content describes the intensity of the crystalline signal to the total scattered intensity. (User Manual for DIFFRAC.EVA, Bruker AXS GmbH, Karlsruhe.)

### REFERENCE EXAMPLE 8: DETERMINATION OF THE C VALUE (BET C CONSTANT)

The C value was determined by usual calculation ((slope/intercept)+1) based on the plot of the BET value 1/(V((p/p₀)−1)) against p/p₀, as known by the skilled person. p is the partial vapour pressure of adsorbate gas in equilibrium with the surface at 77.4 K (b.p. of liquid nitrogen), in Pa, p₀ is the saturated pressure of adsorbate gas, in Pa, and V is the volume of gas adsorbed at standard temperature and pressure (STP) [273.15 K and atmospheric pressure (1.013×10⁵ Pa)], in mL.

### REFERENCE EXAMPLE 9: DETERMINATION OF THE PROPYLENE OXIDE ACTIVITY AND THE PRESSURE DROP RATE (PO TEST)

In the PO test, a preliminary test procedure to assess the possible suitability of the moldings as catalyst for the epoxidation of propene, the moldings were tested in a glass autoclave by reaction of propene with an aqueous hydrogen peroxide solution (30 weight-%) to yield propylene oxide. In particular, 0.5 g of the molding were introduced together with 45 mL of methanol in a glass autoclave, which was cooled to −25° C. 20 mL of liquid propene were pressed into the glass autoclave and the glass autoclave was heated to 0° C. At this temperature, 18 g of an aqueous hydrogen peroxide solution (30 weight-% in water) were introduced into the glass autoclave. After a reaction time of 5 h at 0° C., the mixture was heated to room temperature and the liquid phase was analyzed by gas chromatography with respect to its propylene oxide content. The propylene oxide content of the liquid phase (in weight-%) is the result of the PO test, i.e. the propylene oxide activity of the molding. The pressure drop rate was determined following the pressure progression during the PO test described above. The pressure progression was recorded using a S-11 transmitter (from Wika Alexander Wiegand SE & Co. KG), which was positioned in the pressure line of the autoclave, and a graphic plotter Buddeberg 6100A. The respectively obtained data were read out and depicted in a pressure progression curve. The pressure drop rate (PDR) was determined according to the following equation:

PDR=[p(max)−p(min)]/delta t, with

PDR/(bar/min)=pressure drop rate

p(max)/bar=maximum pressure at the start of the reaction

p(min)/bar=minimum pressure observed during the reaction

delta t/min=time difference from the start of the reaction to the point in time where p(min) was observed

### REFERENCE EXAMPLE 10: DETERMINATION OF THE PROPYLENE EPOXIDATION CATALYTIC PERFORMANCE

In a continuous epoxidation reaction setup, a vertically arranged tubular reactor (length: 1.4 m, outer diameter 10 mm, internal diameter: 7 mm) equipped with a jacket for thermostatization was charged with 15 g of the moldings in the form of strands as described in the respective examples below. The remaining reactor volume was filled with inert material (steatite spheres, 2 mm in diameter) to a height of about 5 cm at the lower end of the reactor and the remainder at the top end of the reactor. Through the reactor, the starting materials were passed with the following flow rates: methanol (49 g/h); hydrogen peroxide (9 g/h; employed as aqueous hydrogen peroxide solution with a hydrogen peroxide content of 40 weight-%); propylene (7 g/h; polymer grade). Via the cooling medium passed through the cooling jacket, the temperature of the reaction mixture was adjusted so that the hydrogen peroxide conversion, determined on the basis of the reaction mixture leaving the reactor, was essentially constant at 90%. The pressure within the reactor was held constant at 20 bar(abs), and the reaction mixture—apart from the fixed-bed catalyst—consisted of one single liquid phase. The reactor effluent stream downstream the pressure control valve was collected, weighed and analyzed. Organic components were analyzed in two separate gas-chromatographs. The hydrogen peroxide content was determined colorimetrically using the titanyl sulfate method. The selectivity for propylene oxide given was determined relative to propene and hydrogen peroxide), and was calculated as 100 times the ratio of moles of propylene oxide in the effluent stream divided by the moles of propene or hydrogen peroxide in the feed.

### REFERENCE EXAMPLE 11: DETERMINATION OF THE TORTUOSITY PARAMETER RELATIVE TO WATER

The tortuosity parameter was determined as described in the experimental section of US 20070099299 A1. In particular, the NMR analyses to this effect were conducted at 25° C. and 1 bar at 125 MHz 1 H resonance frequency with the FEGRIS NT NMR spectrometer (cf. Stallmach et al. in Annual Reports on NMR Spectroscopy 2007, Vol. 61, pp. 51-131). The pulse program used for the PFG NMR self-diffusion analyses was the stimulated spin echo with pulsed field gradients according to FIG. 1b of US 20070099299 A1. For each sample, the spin echo attenuation curves were measured at different diffusion times (between 7 and 100 ms) by stepwise increase in the intensity of the field gradients (to a maximum gmax=10 T/m). From the spin echo attenuation curves, the time dependence of the self-diffusion coefficient of the pore water was determined by means of equations (5) and (6) of US 20070099299 A1. Calculation of the Tortuosity: Equation (7) of US 20070099299 A1 was used to calculate the time dependency of the mean quadratic shift [symbol] z²(Δ) [symbol] =⅓ [symbol] r²(Δ) [symbol] from the self-diffusion coefficients D(Δ) thus determined. By way of example, in FIG. 2 of US 20070099299 A1, the data is plotted for exemplary catalyst supports of said document in double logarithmic form together with the corresponding results for free water. FIG. 2 of US 20070099299 A1 also shows in each case the best fit straight line from the linear fitting of [symbol] r²(Δ) [symbol] as a function of the diffusion time Δ. According to equation (7) of US 2007/0099299 A1, its slope corresponds precisely to the value 6D̅ where D̅ corresponds to the self-diffusion coefficient averaged over the diffusion time interval. According to equation (3) of US 20070099299 A1, the tortuosity is then obtained from the ratio of the mean self-diffusion coefficient of the free solvent (D0) thus determined to the corresponding value of the mean self-diffusion coefficient in the molding.

### REFERENCE EXAMPLE 12: DETERMINATION OF THE PLASTICITY

The plasticity as referred to in the context of the present invention is to be understood as determined via a table-top testing machine Z010/TN2S, supplier Zwick, D-89079 Ulm, Germany. As to fundamentals of this machine and its operation, reference is made to the respective instructions handbook “Betriebsanleitung der Material-Prüfmaschine”, version 1.1, by Zwick Technische Dokumentation, August-Nagel-Strasse 11, D-89079 Ulm, Germany (1999). The Z010 testing machine was equipped with a fixed horizontal table on which a steel test vessel was positioned comprising a cylindrical compartment having an internal diameter of 26 mm and an internal height of 75 mm. This vessel was filled with the composition to be measured so that the mass filled in the vessel did not contain air inclusions. The filling level was 10 mm below the upper edge of the cylindrical compartment. Centered above the cylindrical compartment of the vessel containing the composition to be measured was a plunger having a spherical lower end, wherein the diameter of the sphere was 22.8 mm, and which was freely movable in vertical direction. Said plunger was mounted on the load cell of the testing machine having a maximum test load of 10 kN. During the measurement, the plunger was moved vertically downwards, thus plunging into the composition in the test vessel. Under testing conditions, the plunger was moved at a preliminary force (Vorkraft) of 1.0 N, a preliminary force rate (Vorkraftgeschwindigkeit) of 100 mm/min and a subsequent test rate (Prüfgeschwindigkeit) of 14 mm/min. A measurement was terminated when the measured force reached a value of less than 70% of the previously measured maximum force of this measurement. The experiment was controlled by means of a computer which registered and evaluated the results of the measurements. The maximum force (F_max in N) measured corresponds to the plasticity referred to in the context of the present invention.

### EXAMPLE 1: PROVIDING PARTICLES OF A ZEOLITIC MATERIAL HAVING FRAMEWORK TYPE MFI

A titanium silicalite-1 (TS-1) powder was prepared according to the following recipe: TEOS (tetraethyl orthosilicate) (300 kg) were loaded into a stirred tank reactor at room temperature and stirring (100 r.p.m.) was started. In a second vessel, 60 kg TEOS and 13.5 kg TEOT (tetraethyl orthotitanate) were first mixed and then added to the TEOS in the first vessel. Subsequently, another 360 kg TEOS were added to the mixture in the first vessel. Then, the content of the first vessel was stirred for 10 min before 950 g TPAOH (tetrapropylammonium hydroxide) were added. Stirring was continued for 60 min. Ethanol released by hydrolysis was separated by distillation at a bottoms temperature of 95° C. 300 kg water were then added to the content of the first vessel, and water in an amount equivalent to the amount of distillate was further added. The obtained mixture was stirred for 1 h. Crystallization was performed at 175° C. within 12 h at autogenous pressure. The obtained titanium silicalite-1 crystals were separated, dried, and calcined at a temperature of 500° C. in air for 6 h. The obtained particles of the zeolitic material exhibited a Ti content of 1.9 weight-%, calculated as elemental Ti.

### EXAMPLE 2: PREPARING a MOLDING USING A COLLOIDAL SILICA BINDER PRECURSOR WITH A PARTICLE SIZE DISTRIBUTION ACCORDING TO THE INVENTION

Shaping: The particles of the zeolitic material of Example 1 (105.3 g) and carboxymethyl cellulose (4.0 g; Walocel™, Mw=15,000 g) were mixed in a kneader for 5 min. Then, an aqueous polystyrene dispersion (100.7 g; 33.7 g polystyrene) was continuously added. After 10 min, polyethylene oxide (1.33 g) was added. After 10 min, an aqueous colloidal silica binder precursor (70 g; 50 weight-% SiO₂; Dv10=51 nm; Dv50=72 nm; Dv90=111; from Nalco Chemical Co.) was added. After a further 10 min, 10 mL water were added, after further 5 min additional 10 mL water. The total kneading time was 40 min. The resulting formable mass obtained from kneading, having a plasticity of 1283 N, was extruded at a pressure of 130 bar through a matrix having circular holes with a diameter of 1.9 mm. The obtained strands were dried in air in an oven at a temperature of 120° C. for 4 h and calcined in air at a temperature of 490° C. for 5 h. The crushing strength of the strands determined as described hereinabove was 1.4 N.

Water treatment: 36 g of these strands were mixed in four portions of each 9 g with 180 g deionized water per portion. The resulting mixtures were heated to a temperature of 145° C. for 8 h in an autoclave. Thereafter, the obtained water-treated strands were separated and sieved over a 0.8 mm sieve. The obtained strands were then washed with deionized water and subjected to a stream of nitrogen at ambient temperature. The respectively washed strands were subsequently dried in air at a temperature of 120° C. for 4 h and then calcined in air at a temperature of 450° C. for 2 h.

The resulting material had a TOC of less than 0.1 g/100 g, a Si content of 44 g/100 g, and a Ti content of 1.4 g/100 g. The crushing strength of the strands determined as described hereinabove was 8 N, and the total pore volume determined as described hereinabove was 0.83 mL/g. The tortuosity parameter relative to water was 1.60. The BET specific surface area was 356 m²/g, the C value was −356.

### EXAMPLE 3: PREPARING A MOLDING USING A COLLOIDAL SILICA BINDER PRECURSOR WITH A PARTICLE SIZE DISTRIBUTION ACCORDING TO THE INVENTION

Shaping: The particles of the zeolitic material of Example 1 (105.3 g) and carboxymethyl cellulose (4.0 g; Walocel™, Mw=15,000 g) were mixed in a kneader for 5 min. Then, an aqueous polystyrene dispersion (100.7 g; 33.7 g polystyrene) was continuously added. After 10 min, polyethylene oxide (1.33 g) was added. After 10 min, an aqueous colloidal silica binder precursor (70 g; 40 weight-% SiO₂; Dv10=68 nm; Dv50=97 nm; Dv90=151 nm; from Nalco Chemical Co.) was added. After a further 10 min, 20 mL water were added. The total kneading time was 35 min. The resulting formable mass obtained from kneading was extruded at a pressure of 150 bar through a matrix having circular holes with a diameter of 1.9 mm. The obtained strands were dried in air in an oven at a temperature of 120° C. for 4 h and calcined in air at a temperature of 490° C. for 5 h. The crushing strength of the strands determined as described hereinabove was 1.0 N.

Water treatment: 36 g of these strands were mixed in four portions of each 9 g with 180 g deionized water per portion. The resulting mixtures were heated to a temperature of 145° C. for 8 h in an autoclave. Thereafter, the obtained water-treated strands were separated and sieved over a 0.8 mm sieve. The obtained strands were then washed with deionized water and subjected to a stream of nitrogen at ambient temperature. The respectively washed strands were subsequently dried in air at a temperature of 120° C. for 4 h and then calcined in air at a temperature of 450° C. for 2 h.

The resulting material had a TOC of less than 0.1g/100 g, a Si content of 44 g/100 g, and a Ti content of 1.4. g/100 g. The crushing strength of the strands determined as described hereinabove was 11 N, and the total pore volume determined as described hereinabove was 0.84 mL/g. The tortuosity parameter relative to water was 1.71. The BET specific surface area was 352 m²/g, the C value was −500.

### EXAMPLE 4: PREPARING A MOLDING USING A COLLOIDAL SILICA BINDER PRECURSOR WITH A PARTICLE SIZE DISTRIBUTION ACCORDING TO THE INVENTION

Shaping: The particles of the zeolitic material of Example 1 (105.3 g) and carboxymethyl cellulose (4.0 g; Walocel™, Mw=15,000 g) were mixed in a kneader for 5 min. Then, an aqueous polystyrene dispersion (100.7 g; 33.7 g polystyrene) was continuously added. After 10 min, polyethylene oxide (1.33 g) was added. After 10 min, an aqueous colloidal silica binder precursor (70 g; 50 weight-% SiO₂; Dv10=56; Dv50=81 nm; Dv90=129 nm; from Nalco Chemical Co.) was added. After a further 10 min, 20 mL water were added. The total kneading time was 35 min. The resulting formable mass obtained from kneading was extruded at a pressure of 150 bar through a matrix having circular holes with a diameter of 1.9 mm. The obtained strands were dried in air in an oven at a temperature of 120° C. for 4 h and calcined in air at a temperature of 490° C. for 5 h. The crushing strength of the strands determined as described hereinabove was 1.5 N.

Water treatment: 36 g of these strands were mixed in four portions of each 9 g with 180 g deionized water per portion. The resulting mixtures were heated to a temperature of 145° C. for 8 h in an autoclave. Thereafter, the obtained water-treated strands were separated and sieved over a 0.8 mm sieve. The obtained strands were then washed with deionized water and subjected to a stream of nitrogen at ambient temperature. The respectively washed strands were subsequently dried in air at a temperature of 120° C. for 4 h and then calcined in air at a temperature of 450° C. for 2 h.

The resulting material had a TOC of less than 0.1 g/100 g, a Si content of 44 g/100 g, and a Ti content of 1.4 g/100 g. The crushing strength of the strands determined as described hereinabove was 12 N, and the total pore volume determined as described hereinabove was 0.82 mL/g. The tortuosity parameter relative to water was 1.67. The BET specific surface area was 353 m²/g, the C value was −395.

### Comparative Example 1: Preparing a Molding using a Colloidal Silica Binder Precursor with a Particle Size Distribution not According to the Invention

Shaping: The particles of the zeolitic material of Example 1 (105.3 g) and carboxymethyl cellulose (4.0 g; Walocel™, Mw=15,000 g) were mixed in a kneader for 5 min. Then, an aqueous polystyrene dispersion (100.7 g; 33.7 g polystyrene) was continuously added. After 10 min, polyethylene oxide (1.33 g) was added. After 10 min, an aqueous colloidal silica binder precursor (70 g; 40 weight-% SiO₂; Dv10=28 nm; Dv50=37 nm; Dv90=52 nm; Ludox® AS-40) was added. After a further 10 min, 20 mL water were added. The total kneading time was 35 min. The resulting formable mass obtained from kneading, having a plasticity of 3321 N, was extruded at a pressure of 100 bar through a matrix having circular holes with a diameter of 1.9 mm. The obtained strands were dried in air in an oven at a temperature of 120° C. for 4 h and calcined in air at a temperature of 490° C. for 5 h. The crushing strength of the strands determined as described hereinabove was 1.6 N.

Water treatment: 36 g of these strands were mixed in four portions of each 9 g with 180 g deionized water per portion. The resulting mixtures were heated to a temperature of 145° C. for 8 h in an autoclave. Thereafter, the obtained water-treated strands were separated and sieved over a 0.8 mm sieve. The obtained strands were then washed with deionized water and subjected to a stream of nitrogen at ambient temperature. The respectively washed strands were subsequently dried in air at a temperature of 120° C. for 4 h and then calcined in air at a temperature of 450° C. for 2 h.

The resulting material had a TOC of less than 0.1 g/100 g, a Si content of 44 g/100 g, and a Ti content of 1.5 g/100 g. The crushing strength of the strands determined as described hereinabove was 5 N, and the total pore volume determined as described hereinabove was 0.89 mL/g. The tortuosity parameter relative to water was 1.73. The BET specific surface area was 389 m²/g, the C value was −547.

### Summary of the Crushing Strength Values

In the following Table 1, the crushing strength values of the moldings as prepared above are summarized. Obviously, the moldings of the present invention exhibit significantly higher and therefore highly advantageous values. Moreover, as can be derived from the table, the improvement of the crushing strength values achieved by the water treatment according to step (v) of the process of the invention is significantly better than the respective improvement as regards the process of the prior art.

TABLE 1
Results for catalytic testing according to Reference Example 9

| | crushing | crushing | |
|---|---|---|---|
| | strength/N | strength/N | |
| Molding | (non water- | (water- | improvement/ |
| according to # | treated) | treated) | (100%) *⁾ |
| Example 2 | 1.4 | 8 | +4.7 |
| Example 3 | 1.0 | 11 | +10.0 |
| Example 4 | 1.5 | 12 | +7.0 |
| Comparative | 1.6 | 5 | +2.1 |
| Example 1 | | | |

*⁾ improvement of the crushing strength from non-water treated molding to water-treated molding

### EXAMPLE 5: TESTING THE MOLDINGS AS CATALYSTS FOR EPOXIDIZING PROPENE

### Example 5.1: Preliminary Test—PO Test

Moldings of the examples were preliminarily tested with respect to their general suitability as expoxidation catalysts according to the PO test as described in Reference Example 9. The respective resulting values of the propylene oxide activity are shown in Table 2 below.

TABLE 2
Results for catalytic testing according to Reference Example 9

| Molding | propylene oxide | pressure drop |
|---|---|---|
| according to # | activity/% | rate/(bar/min) |
| Example 2 | 5.0 | 0.013 |
| Example 3 | 4.8 | 0.008 |
| Example 4 | 4.7 | 0.011 |
| Comparative | 4.7 | 0.021 |
| Example 1 | | |

Obviously, the moldings according to the present invention exhibit a very good propylene oxide activity according to the PO test and are promising candidates for catalysts in industrial continuous epoxidation reactions.

### Example 5.2: Catalytic Characteristics of the Moldings in a Continuous Epoxidation Reaction

The characteristics of moldings of the present invention were compared with moldings of the prior art in a continuous epoxidation reaction as described in Reference Example 10. After a significant time on stream (TOS), the hydrogen peroxide conversions of the moldings according to Example 3 and 4 were compared with the respective moldings according to the prior art (Comparative Examples 1).The following results according to Table 3 were obtained:

TABLE 3
Results for catalytic testing according to Reference Example 10

| Molding | | T (cooling | hydrogen peroxide |
|---|---|---|---|
| according to | TOS/h | medium)/° C. | conversion/% |
| Example 3 | 500 | 54 | 95 ± 2 |
| Example 4 | 385 | 55 | 90 ± 2 |
| Comparative | 500 | 56 | 91 ± 2 |
| Example 1 | | | |

### CITED LITERATURE

US 2016/250624 A1
U.S. Pat. No. 6,551,546 B1
DE 19859561 A1
U.S. Pat. No. 7,825,204 B2

1.-20. (canceled)

21. A chemical molding comprising a zeolitic material which exhibits a type I nitrogen adsorption/desorption isotherm and which has framework type MFI and a framework structure comprising Si, O, and Ti, the molding further comprising a binder for said zeolitic material, the binder comprising Si and O, wherein the molding exhibits a total pore volume of at least 0.4 mL/g and a crushing strength of at least 6 N.

22. The molding of claim 21, wherein from 95 to 100 weight-% of the zeolitic material comprised in the molding consist of Si, O, Ti and optionally H, and wherein the zeolitic material comprises Ti in an amount in the range of from 0.2 to 5 weight-%, calculated as elemental Ti and based on the total weight of the zeolitic material.

23. The molding of claim 21, wherein from 95 to 100 weight-% of the binder comprised in the molding consist of Si and O, and wherein the molding comprises the binder, calculated as SiO₂, in an amount in the range of from 2 to 90 weight-% based on the total weight of the molding.

24. The molding of claim 21, wherein from 95 to 100 weight-% of the molding consist of the zeolitic material and the binder.

25. The molding of claim 21, exhibiting a total pore volume in the range of from 0.4 to 1.5 mL/g, and exhibiting a crushing strength in the range of from 6 to 25 N.

26. The molding of claim 21, exhibiting one or more of the following characteristics:
a tortuosity parameter relative to water in the range of from 1.0 to 2.5, determined as described in Reference Example 11;
a BET specific surface area in the range of from 300 to 450 m²/g, determined as described in Reference Example 6;
a crystallinity in the range of from 50 to 100%, determined as described in Reference Example 7;
a propylene oxide activity of at least 4.5 weight-%, determined as described in Reference Example 9;
a pressure drop rate in the range of from 0.005 to 0.019 bar(abs)/min, determined as described in Reference Example 9;
a hydrogen peroxide conversion in the range of from 90 to 95% when used as catalyst in a reaction for preparing propylene oxide from propene and hydrogen peroxide, determined in a continuous epoxidation reaction as described in Reference Example 10 at a temperature of the cooling medium in the range of from 55 to 56° C. at a time on stream in the range of from 200 to 600 hours, wherein the term “time on stream” refers to the duration of the continuous epoxidation reaction without regeneration of the catalyst.

27. A process for preparing a chemical molding comprising a zeolitic material which exhibits a type I nitrogen adsorption/desorption isotherm determined as described in Reference Example 1, and which has framework type MFI and a framework structure comprising Si, O, and Ti, the molding further comprising a binder for said zeolitic material, the binder comprising Si and O, for preparing a chemical molding according to claim 21, the process comprising
(i) providing a zeolitic material exhibiting a type I nitrogen adsorption/desorption isotherm determined as described in Reference Example 1, having framework type MFI and a framework structure comprising Si, O, and Ti;
(ii) providing a binder precursor comprising a colloidal dispersion of silica in water, said binder precursor exhibiting a volume-based particle size distribution characterized by a Dv10 value of at least 35 nanometer, a Dv50 value of at least 45 nanometer, and a Dv90 value of at least 65 nanometer, determined as described in Reference Example 5;
(iii) preparing a mixture comprising the zeolitic material provided in (i) and the binder precursor provided in (ii);
(iv) shaping the mixture obtained from (iii), obtaining a precursor of the molding;
(v) preparing a mixture comprising the precursor of the molding obtained from (iv) and water, and subjecting the mixture to a water treatment under hydrothermal conditions, obtaining a water-treated precursor of the molding;
(vi) calcining the water-treated precursor of the molding in a gas atmosphere, obtaining the molding.

28. The process of claim 27, wherein the volume-based particle size distribution of the colloidal dispersion of silica in water according to (ii) is characterized by a Dv10 value in the range of from 35 to 80 nanometer, a Dv50 value in the range of from 45 to 125 nanometer, and a Dv90 value in the range of from 65 to 200 nanometer, determined as described in Reference Example 5, wherein from 95 to 100 weight-% of the binder precursor according to (ii) consist of the colloidal dispersion of silica in water.

29. The process of claim 27, wherein in the mixture prepared according to (iii) and subjected to (iv), the weight ratio of the zeolitic material, relative to the sum of the zeolitic material and the binder calculated as SiO₂, is in the range of from 2 to 90%, wherein the mixture prepared according to (iii) and subjected to (iv) further comprises one or more additives, one or more viscosity modifying agents, or one or more mesopore forming agents, or one or more viscosity modifying agents and one or more mesopore forming agents, wherein the one or more additives are selected from the group consisting of water, alcohols, organic polymers, and mixtures of two or more thereof, wherein the organic polymers are selected from the group consisting of celluloses, cellulose derivatives, starches, polyalkylene oxides, polystyrenes, polyacrylates, polymethacrylates, polyolefins, polyamides, polyesters, and mixtures of two or more thereof, wherein the organic polymers are more selected from the group consisting of cellulose ethers, polyalkylene oxides, polystyrenes, and mixtures of two or more thereof, wherein the organic polymers are more selected from the group consisting of a methyl celluloses, carboxymethyl celluloses, polyethylene oxides, polystyrenes, and mixtures of two or more thereof.

30. The process of claim 29, wherein in the mixture prepared according to (iii) and subjected to (iv)
the weight ratio of the zeolitic material, relative to the one or more additives, is in the range of from 0.3:1 to 1:1;
the weight ratio of the zeolitic material, relative to the cellulose derivative, is in the range of from 10:1 to 53:1;
the weight ratio of the zeolitic material, relative to the polyethylene oxide, is in the range of from 70:1 to 110:1;
the weight ratio of the zeolitic material, relative to the polystyrene, is in the range of from 2:1 to 8:1;
the weight ratio of the zeolitic material, relative to the water, is in the range of from 0.7:1 to 0.85:1;
wherein the mixture obtained from (iii) and subjected to (iv) has a plasticity in the range of from 500 to 3000 N, determined as described in Reference Example 12.

31. The process of claim 27, wherein shaping according to (iv) further comprises drying the precursor of the molding in a gas atmosphere, wherein said drying is carried out at a temperature of the gas atmosphere in the range of from 80 to 160° C., wherein the gas atmosphere comprises nitrogen, oxygen, or a mixture thereof, wherein the gas atmosphere is oxygen, air, or lean air, and wherein shaping according to (iv) further comprises calcining the dried precursor of the molding in a gas atmosphere, wherein calcining is carried out at a temperature of the gas atmosphere in the range of from 450 to 530° C., wherein the gas atmosphere comprises nitrogen, oxygen, or a mixture thereof, wherein the gas atmosphere is more oxygen, air, or lean air.

32. The process of claim 27, wherein in the mixture prepared in (v), the weight ratio of the precursor of the molding relative to the water is in the range of from 1:1 to 1:30, wherein from 95 to 100 weight-% of the mixture prepared according to (v) consist of the precursor of the molding and water.

33. The process of claim 27, wherein the water treatment according to (v) comprises a temperature of the mixture in the range of from 100 to 200° C., wherein the water treatment according to (v) is carried out under autogenous pressure.

34. The process of claim 27, wherein (v) further comprises separating the water-treated precursor of the molding from the mixture obtained from the water treatment, said separating comprising subjecting the mixture obtained from the water treatment to solid-liquid separation, washing the separated precursor, and drying the washed precursor, wherein said drying according to (v) comprises drying the precursor in a gas atmosphere, wherein drying is carried out at a temperature of the gas atmosphere in the range of from 80 to 160° C. wherein the gas atmosphere comprises nitrogen, oxygen, or a mixture thereof.

35. The process of claim 27, wherein calcining according to (vi) is carried out at a temperature of the gas atmosphere in the range of from 400 to 490° C., wherein the gas atmosphere comprises nitrogen, oxygen, or a mixture thereof.

36. A chemical molding comprising particles of a zeolitic material exhibiting a type I nitrogen adsorption/desorption isotherm determined as described in Reference Example 1, having framework type MFI and a framework structure comprising Si, O, and Ti, the molding further comprising a binder for said particles, the binder the chemical molding according to claim 21.

37. A method comprising utilizing the molding according to claim 21 as an adsorbent, an absorbent, a catalyst or a catalyst component.

38. A method comprising utilizing a colloidal dispersion of silica in water as a binder precursor for preparing a chemical molding comprising a zeolitic material which exhibits a type I nitrogen adsorption/desorption isotherm determined as described in Reference Example 1, and which has framework type MFI and a framework structure comprising Si, O, and Ti, the molding further comprising a binder resulting from said binder precursor, for preparing a molding according to claim 21, said silica exhibiting a volume-based particle size distribution characterized by a Dv10 value of at least 35 nanometer, a Dv50 value of at least 45 nanometer, and a Dv90 value of at least 65 nanometer, said molding exhibiting a total pore volume of at least 0.4 mL/g, and a crushing strength of at least 6 N

39. A mixture comprising a zeolitic material which exhibits a type I nitrogen adsorption/desorption isotherm determined as described in Reference Example 1, and which has framework type MFI and a framework structure comprising Si, O, and Ti, the mixture further comprising a colloidal dispersion of silica in water, said binder precursor exhibiting a volume-based particle size distribution characterized by a Dv10 value of at least 35 nanometer, a Dv50 value of at least 45 nanometer, and a Dv90 value of at least 65 nanometer, said mixture having a plasticity in the range of from 500 to 3000 N, wherein the colloidal dispersion of silica in water comprises the silica in an amount in the range of from 25 to 65 weight-%, based on the total weight of the silica and the water and wherein from 95 to 100 weight-% of the binder precursor consist of the colloidal dispersion of silica in water, wherein in said mixture, the weight ratio of the zeolitic material, relative to the sum of the zeolitic material and the binder calculated as SiO₂, is in the range of from 2 to 90%, wherein said mixture further comprises one or more additives

40. A method comprising utilizing the mixture according to claim 39 for preparing a chemical molding.
